(19) United States
(12) Patent Application Publication
(10) Pub. No.: US 2002/0115669 A1
(43) Pub. Date: Aug. 22, 2002

(54) Oxazolidinone chemotherapeutic agents

(76) Inventors: Paul E. Wiedeman, Deerfield, IL (US); Steven W. Djuric, Libertyville, IL (US); Marina Pilushchev, Vernon Hills, IL (US); Richard J. Sciotti, Saline, IL (US); David J. Madar, Gurnee, IL (US); Hana Kopecka, Vernon Hills, IL (US)

Correspondence Address: Steven F. Weinstock, Abbott Laboratories, Department 377/AP6D-2, 100 Abbott Park Road, Abbott Park, IL, 60064-6050, US

(21) Appl. No.: 09/941,806

(22) Filed: Aug. 29, 2001

Related U.S. Application Data
(60) Provisional application No. 60/229,239, filed on Aug. 31, 2000.

(51) Int. Cl. A61K 31/506; A61K 31/501; A61K 31/4439; A61K 31/655; C07D 413/04; C07D 263/16

(52) U.S. Cl. 514/252.05; 514/256; 514/255.05; 514/340; 514/376; 514/151; 544/238; 544/330; 544/405; 546/271.4; 548/229

(57) ABSTRACT

Compounds of formula (I)

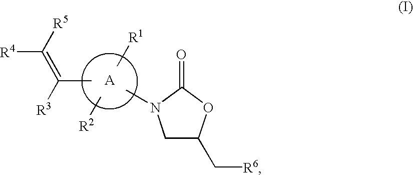

or therapeutically acceptable salts or prodrugs thereof, are useful for treating bacterial infections, psoriasis, arthritis, and toxicity due to chemotherapy. Preparation of the compounds, compositions containing the compounds, and treatment of diseases using the compounds are disclosed.

This application claims priority to co-pending U.S. Provisional Application Ser. No. 60/229,239, filed Aug. 31, 2000.

### TECHNICAL FIELD

This invention is directed to compounds useful for treating bacterial infections, psoriasis, arthritis, and toxicity due to chemotherapy; preparation of the compounds; chemotherapeutic compositions comprising the compounds; and methods for treating diseases using the compounds.

### BACKGROUND OF THE INVENTION

Resistance to antibiotics once useful for treatment of bacterial infections resulting from pathogens such as Staphylococcus aureus, Staphylococcus epidermidis, and Enterococcus faecium has become a significant problem (Drugs Exp. Clin. Res. 1994, XX, 215-224; Am. J. Surg. 1995, 5A (Suppl.), 8S-12S; Drugs, 1994, 48, 678-688; and Current Pharmaceutical Design, 1996, Vol.2, No.2, pp175-194). Thus, the development of new broad spectrum synthetic and semi-synthetic antibacterial compounds is the subject of constant current research.

One such class is synthetic oxazolidinones, exemplified by eperezolid and linezolid, which constitute a class of orally active, antibacterial agents with good activity against Gram-positive bacteria (Current Pharmaceutical Design, op. cit.). Reference is also made to U.S. Pat. No. 4,977,173 and European Patent 127,902-B1, each of which teaches a series of antibacterial compounds comprising oxazolidinones connected to a substituted alkene through a phenyl ring.

U.S. Pat. No. 6,040,306, the disclosure of which is hereinafter incorporated by reference into this specification, also teaches the use of oxazolidinones for treatment of psoriasis, arthritis, and toxicity due to chemotherapy.

Given these and other reports on the therapeutic benefit of oxazolidinone antibacterials, the loss of activity among antibacterials which were once efficacious for treatment of certain Gram-positive bacteria, and the continuing need for treatment of diseases such as psoriasis, arthritis, and toxicity due to chemotherapy, there is a continuing need for the development of novel oxazolidinone drugs with modified or improved profiles of activity.

### SUMMARY OF THE INVENTION

In its principle embodiment, therefore, the instant invention is directed to compounds which can be useful for treating bacterial infections, psoriasis, arthritis, and toxicity due to chemotherapy, said compounds having structural formula (I)

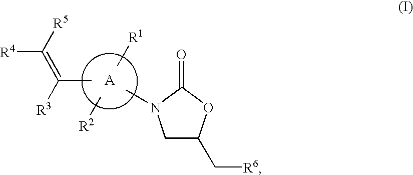

or therapeutically acceptable salts or prodrugs thereof, wherein

A is selected from

(a) phenyl,

(b) a five-membered aromatic ring containing 1-3 atoms selected from N, O, and S, and the remaining atoms are carbon,

wherein the groups defining (b) are substituted on a substitutable carbon or nitrogen atom in the ring, and

(c) a six-membered aromatic ring containing 1-3 nitrogen atoms, and the remaining atoms are carbon;

wherein the groups defining (c) are substituted on a substitutable carbon atom in the ring;

R¹ and R² are independently selected from hydrogen, alkyl, alkoxy, thioalkoxy, cycloalkyl, hydroxy, amino, aminoalkyl, halo, haloalkyl, and perfluoroalkyl;

R³, R⁴, and R⁵ are independently selected from

(a) hydrogen,

(b) carboxamido, cyano, halo, nitro, perfluoroalkyl,

(c) alkyl, alkanoyl, cycloalkyl, cyclothioalkoxy, cycloalkylsulfinyl, cycloalkoxycarbonyl, thioalkoxy, alkylsulfinyl, alkylsulfonyl, alkoxycarbonyl, cycloalkenyl, thiocycloalkenyloxy, cycloalkenylsulfinyl, cycloalkenylsulfonyl,

wherein the groups defining (c) can be optionally substituted with 1-5 substituents independently selected from alkoxy, alkanoyloxy, alkoxycarbonyl, amino, azido, carboxamido, carboxy, cyano, halo, hydroxy, nitro, perfluoroalkyl, perfluoroalkoxy, oxo, thioalkoxy, unsubstituted or substituted aryl, unsubstituted or substituted heteroaryl, and unsubstituted or substituted heterocycle,

(d) aryl, arylalkyl, arylthio, arylsulfinyl, arylsulfonyl, aryloxycarbonyl, heteroaryl, heteroarylalkyl, heteroarylsulfinyl, heteroarylsulfonyl, heteroaryloxycarbonyl, heterocycle, (heterocycle)alkyl, (heterocycle)sulfinyl, (heterocycle)sulfonyl, and (heterocycle)oxycarbonyl,

wherein the groups defining (d) can be optionally substituted with 1-5 substituents independently selected from alkyl, alkoxy, alkoxyalkyl, alkoxyalkenyl, alkanoyl, alkanoyloxy, alkanoyloxyalkyl, alkanoyloxyalkenyl, alkoxycarbonyl, alkoxycarbonylalkyl, alkoxycarbonylalkenyl, alkylsulfonyl, alkylsulfonylalkyl, alkylsulfonylalkenyl, amino, aminoalkyl, aminoalkenyl, aminosulfonyl, aminosulfonylalkyl, aminosulfonylalkenyl, azido, carboxaldehyde, (carboxaldehyde)alkyl, (carboxaldehyde)alkenyl, carboxamido, carboxamidoalkyl, carboxamidoalkenyl, carboxy, carboxyalkyl, carboxyalkenyl, cyano, cyanoalkyl, cyanoalkenyl, halo, haloalkyl, haloalkenyl, hydroxy, hydroxyalkyl, hydroxyalkenyl, nitro, oxo, perfluoroalkyl, perfluoroalkoxy, perfluoroalkoxyalkyl, perfluoroalkoxyalkenyl thioalkoxy, thioalkoxyalkyl, thioalkoxyalkenyl, unsubstituted or substituted aryl, unsubstituted or substituted heteroaryl, and unsubstituted or substituted heterocycle,

wherein for the groups defining (c) and (d), the substituted aryl, the substituted heteroaryl, and the substituted heterocycle are substituted with 1-5 substituents independently selected from alkyl, alkoxy, carboxy, azido, carboxaldehyde, halo, hydroxy, cyano, thioalkoxy, amino, alkoxycarbonyl, arylsulfonyl, oxo, perfluoroalkyl, and perfluoroalkoxy; or

wherein R⁴ and R⁵ together are a 3- to 8-membered cycloalkyl;

with the proviso that combinations wherein R³, R⁴, and R⁵ are hydrogen are excluded;

R⁶ is selected from NHR⁷, N-phthalimide (N-Phth), NR⁷R⁸, N(R⁸)C(O)OR⁹, N(R⁸)C(O)N(R⁸)₂, OR⁹, SR⁹, S(O)R⁹, and SO₂R⁹;

R⁷ is selected from alkanoyl, aryloyl, thioalkanoyl, heteroaryl, heteroarylalkyl, (heteroaryl)oyl, heterocycle, and (heterocycle)alkyl,

wherein the groups defining R⁷ can be optionally substituted with 1-5 substituents independently selected from alkyl, alkoxy, alkoxycarbonyl, carboxy, azido, carboxaldehyde, halo, hydroxy, perfluoroalkyl, and perfluoroalkoxy;

R⁸ is selected from

(a) hydrogen,

(b) alkyl,

wherein the alkyl can be optionally substituted with 1-5 substituents independently selected from alkoxy, aryl, alkoxycarbonyl, carboxy, azido, carboxaldehyde, halo, hydroxy, perfluoroalkyl, and perfluoroalkoxy;

(c) cycloalkyl, cycloalkylalkyl, aryl, heteroaryl, heteroarylalkyl, heterocycle, and (heterocycle)alkyl;

wherein the groups defining (c) can be optionally substituted with 1-5 substituents independently selected from alkyl, alkoxy, carboxy, azido, carboxaldehyde, halo, hydroxy, perfluoroalkyl, and perfluoroalkoxy; and

(d) a nitrogen protecting group; and

R⁹ is selected from

(a) alkyl,

wherein the alkyl can be optionally substituted with 1-5 substituents independently selected from alkoxy, carboxy, azido, carboxaldehyde, halo, hydroxy, perfluoroalkyl, and perfluoroalkoxy,

(b) cycloalkyl, cycloalkylalkyl, aryl, heteroaryl, heteroarylalkyl, heterocycle, and (heterocycle)alkyl;

wherein the groups defining (b) can be optionally substituted with 1-5 substituents independently selected from alkyl, alkoxy, carboxy, azido, carboxaldehyde, halo, hydroxy, perfluoroalkyl, and perfluoroalkoxy;

with the proviso that combinations wherein A is phenyl, R⁶ is NHR⁷ wherein R⁷ is alkanoyl, one of R³, R⁴ or R⁵ is hydrogen, another is selected from hydrogen, perfluoroalkyl, or unsubstituted alkyl, and the remainder is phenyl unsubstituted or substituted with at least one halo substituent, are excluded; and

with the proviso that combinations wherein A is phenyl, R⁶ is NHR⁷ wherein R⁷ is alkanoyl, one of R³, R⁴ or R⁵ is cyano, carboxaldehyde, or nitro, and the remainder are hydrogen or alkyl are excluded.

### DETAILED DESCRIPTION OF THE INVENTION

The compounds of the instant invention are substituted oxazolidinones which can be useful for treating bacterial infections, psoriasis, arthritis, toxicity due to chemotherapy, and obesity. In its principle embodiment, the invention is directed to compounds of formula (I)

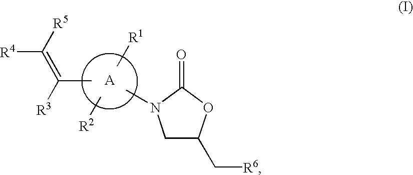

or therapeutically acceptable salts thereof, wherein A, R¹, R², R³, R⁴, R⁵, and R⁶ are defined hereinabove.

The compounds of the invention comprise oxazolidinones connected through the nitrogen atom in the oxazolidinone ring to a substituted alkene through ring A. Ring A is a stable, aromatic, monocyclic group substituted through carbon atoms or nitrogen atoms in the ring. Preferably, ring A is phenyl, although heteroaryl rings such as furanyl, thienyl, pyrrolyl, oxazolyl, thiazolyl, imidazolyl, pyrazolyl, isoxazolyl, isothiazolyl, pyridyl, pyridazinyl, pyrimidinyl, and pyrazinyl are within the scope of the invention. Ring A can be further substituted by independent replacement of one or two hydrogen atoms thereon by substituents defined by R¹ and R² so that, for instance and by way of example only, ring A can be substituted by halo, preferably fluoro. Lines drawn into ring A (such as from R¹ and R²) indicate that the bonds can be attached to any substitutable ring carbon atom.

R³, R⁴, and R⁵ can likewise vary considerably without departing from the intent of the invention. Preferred substituents include, but are not limited to, halo, unsubstituted or substituted alkyl, unsubstituted or substituted aryl, unsubstituted or substituted heteroaryl, unsubstituted or substituted arylsulfonyl, unsubstituted or substituted alkoxycarbonyl, and the like.

It is intended that the definition of any substituent or variable at a particular part in a molecule be independent of its definition elsewhere in that molecule. For example, for substituents defined by R⁸, it is intended that the definition of an R⁸ substituent at one location is independent of its definition elsewhere. Thus, N(R⁸)C(O)N(R⁸)₂ represents, for instance, and by way of example only, N(CH₃)C(O)N(C₂H₅)(C₃H₇), and the like. In a preferred embodiment of the invention, R⁶ is NHR⁷ wherein R⁷ is alkanoyl, preferably acetyl.

Other preferred embodiments of the invention include compounds of formula (I) wherein R³ is hydrogen; compounds of formula (I) wherein R⁴ is hydrogen; compounds of formula (I) wherein R⁶ is —NHR⁷; and compounds of formula (I) wherein R⁷ is alkanoyl.

It is believed that when the compounds have attached thereto a hydroxyl, amino, or carboxylic acid group, prodrugs can be prepared from these compounds by attaching thereto a prodrug-forming group. These prodrugs can then be rapidly transformed in vivo to the parent compound, such as, for example, by hydrolysis in blood. The term “therapeutically acceptable prodrug,” as used herein, refers to those prodrugs of the compounds which are suitable for use in contact with the tissues of humans and lower animals with undue toxicity, irritation, allergic response, and the like, commensurate with a reasonable benefit/risk ratio, and effective for their intended use, as well as the zwitterionic forms, wherein possible, of the compounds.

This invention is based, in part, on the structure activity relationship data provided hereinbelow. Therefore, another embodiment of the invention encompasses any compound, including metabolic precursors of the inhibitor compounds, which contain an essential inhibitory group as disclosed herein. These inhibitory groups can be in masked form or prodrug form and can be released by metabolic or other processes after administration to a patient.

Because asymmetric centers exist in the compounds, the invention contemplates stereoisomers and mixtures thereof. Individual stereoisomers of compounds are prepared by synthesis from starting materials containing the chiral centers or by preparation of mixtures of enantiomeric products followed by separation such as conversion to a mixture of diastereomers followed by separation or recrystallization, chromatographic techniques, or direct separation of the enantiomers on chiral chromatographic columns. Starting compounds of particular stereochemistry are either commercially available or are made by the methods described below and resolved by techniques well-known in the art.

Because carbon-carbon double bonds also exist in the compounds, the invention contemplates various geometric isomers and mixtures thereof resulting from the arrangement of substituents around these carbon-carbon double bonds. These substituents are designated as being in the E or Z configuration wherein the term “E” refers to higher order substituents on opposite sides of the carbon-carbon double bond, and the term “Z” refers to higher order substituents on the same side of the carbon-carbon double bond. A thorough discussion of E and Z isomerism is provided in Advanced Organic Chemistry. Reactions, Mechanisms, and Structure, 4th ed., John Wiley & Sons, New York, 1992, pp. 109-112.

Accordingly, it will be understood by those skilled in the art that another embodiment of compounds of formula (I) are compounds of formula (II)

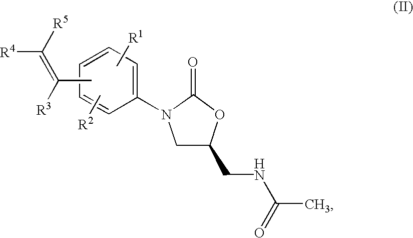

or therapeutically acceptable salts or prodrugs or thereof, wherein R¹, R², R³, R⁴, and R⁵ are defined hereinabove.

In yet another embodiment of the invention is disclosed a composition comprising a compound of formula (I), or a therapeutically acceptable salt or prodrug thereof, and a therapeutically acceptable excipient.

In yet another embodiment of the invention is disclosed a composition comprising a compound of formula (II), or a therapeutically acceptable salt or prodrug thereof, and a therapeutically acceptable excipient.

In yet another embodiment of the invention is disclosed a method for treating bacterial infections in a patient comprising administering to the patient a therapeutically acceptable amount of a compound of formula (I) or a therapeutically acceptable salt or prodrug thereof.

In yet another embodiment of the invention is disclosed a method for treating bacterial infections in a patient comprising administering to the patient a therapeutically acceptable amount of a compound of formula (II) or a therapeutically acceptable salt or prodrug thereof.

In yet another embodiment of the invention is disclosed a method for treating psoriasis in a patient comprising administering to the patient a therapeutically acceptable amount of a compound of formula (I) or a therapeutically acceptable salt or prodrug thereof.

In yet another embodiment of the invention is disclosed a method for treating psoriasis in a patient comprising administering to the patient a therapeutically acceptable amount of a compound of formula (II) or a therapeutically acceptable salt or prodrug thereof.

In yet another embodiment of the invention is disclosed a method for treating arthritis in a patient comprising administering to the patient a therapeutically acceptable amount of a compound of formula (I) or a therapeutically acceptable salt or prodrug thereof.

In yet another embodiment of the invention is disclosed a method for treating arthritis in a patient comprising administering to the patient a therapeutically acceptable amount of a compound of formula (II) or a therapeutically acceptable salt or prodrug thereof.

In yet another embodiment of the invention is disclosed a method for treating toxicity due to chemotherapy in a patient comprising administering to the patient a therapeutically acceptable amount of a compound of formula (I) or a therapeutically acceptable salt or prodrug thereof.

In yet another embodiment of the invention is disclosed a method for treating toxicity due to chemotherapy in a patient comprising administering to the patient a therapeutically acceptable amount of a compound of formula (II) or a therapeutically acceptable salt or prodrug thereof.

As used throughout the specification, the following terms have the meanings indicated:

The term “alkanoyl,” as used herein, refers to an alkyl group, as defined herein, attached to the parent molecular group through a carbonyl group.

The term “alkanoyloxy,” as used herein, refers to an alkanoyl group, as defined herein, attached to the parent molecular group through an oxygen atom.

The term “alkanoyloxyalkyl,” as used herein, refers to an alkyl group, as defined herein, to which is attached at least one alkanoyloxy substituent.

The term “alkanoyloxyalkenyl,” as used herein, refers to an alkenyl group, as defined herein, to which is attached at least one alkanoyloxy substituent.

The term “alkenyl,” as used herein, refers to a monovalent straight or branched chain hydrocarbon having from two to six carbons and at least one carbon-carbon double bond.

The term “alkoxy,” as used herein, refers to an alkyl group, as defined herein, attached to the parent molecular group through an oxygen atom.

The term “alkoxyalkyl,” as used herein, refers to an alkyl group, as defined herein, to which is attached at least one alkoxy substituent.

The term “alkoxyalkenyl,” as used herein, refers to an alkenyl group, as defined herein, to which is attached at least one alkoxy substituent.

The term “alkoxycarbonyl,” as used herein, refers to an alkoxy group, as defined herein, attached to the parent molecular group through a carbonyl group.

The term “alkoxycarbonylalkyl,” as used herein, refers to an alkyl group, as defined herein, to which is attached at least one alkoxycarbonyl substituent.

The term “alkoxycarbonylalkenyl,” as used herein, refers to an alkenyl group, as defined herein, to which is attached at least one alkoxycarbonyl substituent.

The term “alkyl,” as used herein, refers to a saturated, monovalent straight or branched chain hydrocarbon having from one to six carbons. The alkyl groups of this invention can be optionally substituted with 1-5 substituents selected from alkoxy, alkanoyloxy, alkoxycarbonyl, amino, aminoalkyl, azido, carboxamido, carboxy, cyano, halo, hydroxy, nitro, perfluoroalkyl, perfluoroalkoxy, oxo, thioalkoxy, unsubstituted or substituted aryl, unsubstituted or substituted heteroaryl, and unsubstituted or substituted heterocycle. The substituted aryl, substituted heteroaryl, and substituted heterocycle groups substituting the alkyl groups of this invention are substituted with at least one substituent selected from alkyl, alkoxy, amino, aminoalkyl, carboxy, azido, carboxaldehyde, halo, hydroxy, perfluoroalkyl, and perfluoroalkoxy.

The term “alkylsulfinyl,” as used herein, refers to an alkyl group, as defined herein, attached to the parent molecular group through an —S(O)— group.

The term “alkylsulfinylalkyl,” as used herein, refers to an alkyl group, as defined herein, to which is attached at least one alkylsulfinyl substituent.

The term “alkylsulfinylalkenyl,” as used herein, refers to an alkenyl group, as defined herein, to which is attached at least one alkylsulfinyl substituent.

The term “alkylsulfonyl,” as used herein, refers to an alkyl group, as defined herein, attached to the parent molecular group through a sulfonyl group.

The term “alkylsulfonylalkyl,” as used herein, refers to an alkyl group, as defined herein, to which is attached at least one alkylsulfonyl substituent.

The term “alkylsulfonylalkenyl,” as used herein, refers to an alkenyl group, as defined herein, to which is attached at least one alkylsulfonyl substituent.

The term “amino,” as used herein, refers to —NH₂ or derivatives thereof formed by independent replacement of one or both hydrogen atoms thereon with a substituent or substituents independently selected from alkyl, alkanoyl, aryl, arylalkyl, cycloalkyl, cycloalkylalkyl, heteroaryl, heteroarylalkyl, and an amino protecting group.

The “term aminoalkyl,” as used herein, refers to an alkyl group, as defined herein, to which is attached at least one amino substituent.

The term “aminoalkenyl,” as used herein, refers to an alkenyl group, as defined herein, to which is attached at least one amino substituent, spaced apart from the carbon-carbon double bond by at least one sp carbon.

The terms “amino protecting group,” or “nitrogen protecting group,” as used herein, refer to selectively introducible and removable groups which protect amino groups against undesirable side reactions during synthetic procedures. Examples of amino protecting groups include methoxycarbonyl, ethoxycarbonyl, trichloroethoxycarbonyl, benzyloxycarbonyl (Cbz), chloroacetyl, trifluoroacetyl, phenylacetyl, benzoyl (Bn), benzyl (Bz), 2,4-dimethoxybenzyl, tert-butoxycarbonyl (Boc), para-methoxybenzyloxycarbonyl, isopropoxycarbonyl, phthaloyl, succinyl, diphenylmethyl, triphenylmethyl (trityl), methanesulfonyl, para-toluenesulfonyl, trimethylsilyl, triethylsilyl, triphenylsilyl, and the like. Preferred amino or nitrogen protecting groups of this invention are phthalyl and 2,4-dimethoxybenzyl. Amino protecting group can also be used as prodrug-forming groups.

The term “aminosulfonyl,” as used herein, refers to an amino group, as defined herein, attached to the parent molecular group through a sulfonyl group.

The term “arylsulfonylalkyl,” as used herein, refers to an alkyl group, as defined herein, to which is attached at least one arylsulfonyl substituent.

The term “arylsulfonylalkenyl,” as used herein, refers to an alkenyl group, as defined herein, to which is attached at least one arylsulfonyl substituent.

The term “aryl,” as used herein, refers to groups containing at least one aromatic, carbocyclic ring. Aryl groups of this invention are exemplified by phenyl, naphthyl, indenyl, indanyl, dihydronaphthyl, tetrahydronaphthyl, and the like. The aryl groups of this invention can be optionally substituted with 1-5 substituents independently selected from alkyl, alkoxy, alkoxyalkyl, alkoxyalkenyl, alkanoyl, alkanoyloxy, alkanoyloxyalkyl, alkanoyloxyalkenyl, alkoxycarbonyl, alkoxycarbonylalkyl, alkoxycarbonylalkenyl, alkylsulfonyl, alkylsulfonylalkyl, alkylsulfonylalkenyl, amino, aminoalkyl, aminoalkenyl, aminosulfonyl, aminosulfonylalkyl, aminosulfonylalkenyl, arylalkenyl, azido, carboxaldehyde, (carboxaldehyde)alkyl, (carboxaldehyde)alkenyl, carboxamido, carboxamidoalkyl, carboxamidoalkenyl, carboxy, carboxyalkyl, carboxyalkenyl, cyano, cyanoalkyl, cyanoalkenyl, formyl, halo, haloalkyl, haloalkenyl, heteroarylalkenyl, hydroxy, hydroxyalkyl, hydroxyalkenyl, nitro, oxo, perfluoroalkyl, perfluoroalkoxy, perfluoroalkoxyalkyl, perfluoroalkoxyalkenyl thioalkoxy, thioalkoxyalkyl, thioalkoxyalkenyl, unsubstituted or substituted aryl, unsubstituted or substituted heteroaryl, and unsubstituted or substituted heterocycle. The substituted aryl, heteroaryl, and heterocycle groups substituting the aryl groups of this invention are substituted with at least one substituent selected from alkyl, alkoxy, carboxy, azido, carboxaldehyde, halo, hydroxy, perfluoroalkyl, and perfluoroalkoxy.

The term “arylalkyl,” as used herein, refers to an alkyl group, as defined herein, to which is attached at least one aryl substituent. The aryl groups of this radical can be optionally substituted with 1-3 substitutents independently selected from alkyl, alkoxy, carboxy, azido, carboxaldehyde, halo, hydroxy, perfluoroalkyl, and perfluoroalkoxy.

The term “arylalkenyl,” as used herein, refers to an alkenyl group, as defined herein, to which is attached at least one aryl substituent.

The term “aryloyl,” as used herein, refers to an aryl group, as defined herein, attached to the parent molecular group through a carbonyl group.

The term “arylsulfinyl,” as used herein, refers to an aryl group, as defined herein, attached to the parent molecular group through an —S(O)— group.

The term “arylsulfinylalkyl,” as used herein, refers to an alkyl group, as defined herein, to which is attached at least one arylsulfinyl substituent.

The term “arylsulfinylalkenyl,” as used herein, refers to an alkenyl group, as defined herein, to which is attached at least one arylsulfinyl substituent.

The term “arylsulfonyl,” as used herein, refers to an alkyl group, as defined herein, attached to the parent molecular group through a sulfonyl group.

The term “arylsulfonylalkyl,” as used herein, refers to an alkyl group, as defined herein, to which is attached at least one arylsulfonyl substituent.

The term “arylsulfonylalkenyl,” as used herein, refers to an alkenyl group, as defined herein, to which is attached at least one arylsulfonyl substituent.

The term “arylthio,” as used herein, refers to an aryl group, as defined herein, attached to the parent molecular group through a sulfur atom.

The term “azido,” as used herein, refers to —N₃.

The term “carbonyl,” as used herein, refers to —C(═O)—.

The term “carboxamido,” as used herein, refers to an amide; e.g., an amino group, as defined herein, attached to the parent molecular group through a carbonyl group.

The term “carboxamidoalkyl,” as used herein, refers to an alkyl group, as defined herein, to which is attached at least one carboxamido substituent.

The term “carboxamidoalkenyl,” as used herein, refers to an alkenyl group, as defined herein, to which is attached at least one carboxamido substituent.

The term “carboxy,” as used herein, refers to —CO₂H or a derivative thereof formed by replacement of the hydrogen atom thereon by a carboxy protecting group.

The term “carboxyalkyl,” as used herein, refers to an alkyl group, as defined herein, to which is attached at least one carboxy substituent.

The term “carboxyalkenyl,” as used herein, refers to an alkenyl group, as defined herein, to which is attached at least one carboxy substituent.

The term “carboxy protecting group,” as used herein, refers to selectively introducible and removable groups which protect carboxy groups against undesirable side reactions during synthetic procedures and includes all conventional carboxy protecting groups. Examples of carboxy protecting groups include methyl, ethyl, n-propyl, isopropyl, 1,1-dimethylpropyl, n-butyl, tert-butyl, phenyl, naphthyl, benzyl, diphenylmethyl, triphenylmethyl (trityl), para-nitrobenzyl, para-methoxybenzyl, acetylmethyl, benzoylmethyl, para-nitrobenzoylmethyl, para-bromobenzoylmethyl, 2-tetrahydropyranyl 2-tetrahydrofuranyl, 2,2,2-trichloroethyl cyclopropyl, cyclobutyl, cyclopentyl, cyclohexyl, methoxymethyl, methoxyethoxymethyl, arylalkoxyalkyl benzyloxymethyl 1,1-dimethyl-2-propenyl, 3-methyl-3-butenyl, allyl, and the like. Carboxy protecting group can also be used as prodrug-forming groups.

The term “cyano,” as used herein, refers to —CN.

The term “cyanoalkyl,” as used herein, refers to an alkyl group, as defined herein, to which is attached at least one cyano substituent.

The term “cyanoalkenyl,” as used herein, refers to an alkenyl group, as defined herein, to which is attached at least one cyano substituent.

The term “cycloalkenyl,” as used herein, refers to a monovalent cyclic or bicyclic hydrocarbon of three to fifteen carbons and at least one carbon carbon double bond.

The term “cycloalkenylsulfinyl,” as used herein, refers to a cycloalkenyl group, as defined herein, attached to the parent molecular group through a —S(O)— group.

The term “cycloalkenylsulfonyl,” as used herein, refers to a cycloalkenyl group, as defined herein, attached to the parent molecular group through a —S(O)₂— group.

The term “cycloalkoxy,” as used herein, refers to a cycloalkyl group, as defined herein, attached to the parent molecular group through an oxygen atom.

The term “cycloalkoxycarbonyl,” as used herein, refers to an cycloalkoxy group, as defined herein, attached to the parent molecular group through a carbonyl group.

The term “cycloalkyl,” as used herein, refers to a monovalent saturated cyclic or bicyclic hydrocarbon of three to fifteen carbons. The cycloalkyl groups of this invention can be optionally substituted with 1-5 substituents independently selected from alkoxy, alkanoyloxy, alkoxycarbonyl, amino, azido, carboxamido, carboxy, cyano, halo, hydroxy, nitro, perfluoroalkyl, perfluoroalkoxy, oxo, thioalkoxy, unsubstituted or substituted aryl, unsubstituted or substituted heteroaryl, and unsubstituted or substituted heterocycle. The substituted aryl, substituted heteroaryl, and substituted heterocycle substituting the cycloalkyl groups of this invention are substituted with 1-5 substituents independently selected from alkyl, alkoxy, carboxy, azido, carboxaldehyde, halo, hydroxy, perfluoroalkyl, and perfluoroalkoxy.

The term “cycloalkylsulfinyl,” as used herein, refers to a cycloalkyl group, as defined herein, attached to the parent molecular group through an —S(O)— group.

The term “cycloalkylsulfonyl,” as used herein, refers to a cycloalkyl group, as defined herein, attached to the parent molecular group through an —SO₂— group.

The term “cycloalkylalkyl,” as used herein, refers to an alkyl group, as defined herein, to which is attached at least one cycloalkyl substituent.

The term “cycloalkylalkenyl,” as used herein, refers to an alkenyl group, as defined herein, to which is attached at least one cycloalkyl substituent.

The term “cyclothioalkoxy,” as used herein, refers to a cycloalkyl group, as defined herein, attached to the parent molecular group through a sulfur atom.

The term “halo” as used herein, refers to F, Cl, or Br.

The term “haloalkyl,” as used herein, refers to an alkyl group, as defined herein, to which is attached at least one halo substituent.

The term “haloalkenyl,” as used herein, refers to an alkenyl group, as defined herein, to which is attached at least one halo substituent.

The term “heteroaryl,” as used herein, refers to cyclic, aromatic five- and six-membered groups, wherein at least one atom is selected from the group consisting of N, O, and S, and the remaining atoms are carbon. The five-membered rings have two double bonds, and the six-membered rings have three double bonds. The heteroaryl groups of the invention are connected to the parent molecular group through a substitutable carbon or nitrogen in the ring. Heteroaryls are exemplified by furanyl, thienyl, pyrrolyl, oxazolyl, thiazolyl, imidazolyl, isoxazolyl, isothiazolyl, oxadiazolyl, oxadiazolyl, triazolyl, thiadiazolyl, pyridyl, pyridazinyl, pyrimidinyl, pyrazinyl, pyrazolyl, triazinyl, tetrazolyl, and the like. The heteroaryl groups of this invention can be fused to an aryl group, a heterocycle, or another heteroaryl. The heteroaryl groups of this invention can be optionally substituted with 1-5 substituents independently selected from alkyl, alkoxy, alkoxyalkyl, alkoxyalkenyl, alkanoyl, alkanoyloxy, alkanoyloxyalkyl, alkanoyloxyalkenyl, alkoxycarbonyl, alkoxycarbonylalkyl, alkoxycarbonylalkenyl, alkylsulfonyl, alkylsulfonylalkyl, alkylsulfonylalkenyl, amino, aminoalkyl, aminoalkenyl, aminosulfonyl, aminosulfonylalkyl, aminosulfonylalkenyl, arylalkenyl, azido, carboxaldehyde, (carboxaldehyde)alkyl, (carboxaldehyde)alkenyl, carboxamido, carboxamidoalkyl, carboxamidoalkenyl, carboxy, carboxyalkyl, carboxyalkenyl, cyano, cyanoalkyl, cyanoalkenyl, halo, haloalkyl, haloalkenyl, heteroarylalkenyl, hydroxy, hydroxyalkyl, hydroxyalkenyl, nitro, oxo, perfluoroalkyl, perfluoroalkoxy, perfluoroalkoxyalkyl, perfluoroalkoxyalkenyl thioalkoxy, thioalkoxyalkyl, thioalkoxyalkenyl, unsubstituted or substituted aryl, unsubstituted or substituted heteroaryl, and unsubstituted or substituted heterocycle. The substituted aryl, heteroaryl, and heterocycle substituting the heteroaryl groups of this invention are substituted with at least one substituent selected from alkyl, alkoxy, carboxy, azido, carboxaldehyde, halo, hydroxy, perfluoroalkyl, and perfluoroalkoxy.

The term “heteroarylalkyl,” as used herein, refers to an alkyl group, as defined herein, to which is attached at least one heteroaryl substituent.

The term “heteroarylalkenyl,” as used herein, refers to an alkenyl group, as defined herein, to which is attached at least one heteroaryl substituent.

The term “(heteroaryl)oyl,” as used herein, refers to a heteroaryl group, as defined herein, attached to the parent molecular group through a carbonyl.

The term “heterocycle,” as used herein, refers to cyclic, non-aromatic, four-, five-, six-, or seven-membered rings containing at least one atom selected from the group consisting of oxygen, nitrogen, and sulfur. The four-membered rings have zero double bonds, the five-membered rings have zero or one double bonds, and the six- and seven-membered rings have zero, one, or two double bonds. Heterocycle groups of the invention are exemplified by dihydropyridinyl, morpholinyl, phthalimide, piperazinyl, pyrrolidinyl, tetrahydropyridinyl, piperidinyl, thiomorpholinyl, 1,3-dioxolanyl, 1,4-dioxanyl, 1,3-dioxanyl, and the like. The heterocycle groups of this invention can be fused to an aryl group or a heteroaryl group. The heterocycle groups of the invention are connected to the parent molecular group through a substitutable carbon or nitrogen atom in the ring. The heterocycle groups of this invention can be optionally substituted with 1-5 substituents independently selected from alkyl, alkoxy, alkoxyalkyl, alkoxyalkenyl, alkanoyl, alkanoyloxy, alkanoyloxyalkyl, alkanoyloxyalkenyl, alkoxycarbonyl, alkoxycarbonylalkyl, alkoxycarbonylalkenyl, alkylsulfonyl, alkylsulfonylalkyl, alkylsulfonylalkenyl, amino, aminoalkyl, aminoalkenyl, aminosulfonyl, aminosulfonylalkyl, aminosulfonylalkenyl, azido, carboxaldehyde, (carboxaldehyde)alkyl, (carboxaldehyde)alkenyl, carboxamido, carboxamidoalkyl, carboxamidoalkenyl, carboxy, carboxyalkyl, carboxyalkenyl, cyano, cyanoalkyl, cyanoalkenyl, halo, haloalkyl, haloalkenyl, hydroxy, hydroxyalkyl, hydroxyalkenyl, nitro, oxo, perfluoroalkyl, perfluoroalkoxy, perfluoroalkoxyalkyl, perfluoroalkoxyalkenyl thioalkoxy, thioalkoxyalkyl, thioalkoxyalkenyl, unsubstituted or substituted aryl, unsubstituted or substituted heteroaryl, and unsubstituted or substituted heterocycle. The substituted aryl, heteroaryl, and heterocycle groups substituting the heterocycle groups of this invention are substituted with at least one substituent selected from alkyl, alkoxy, carboxy, azido, carboxaldehyde, halo, hydroxy, perfluoroalkyl, and perfluoroalkoxy.

The term “(heterocycle)alkyl,” as used herein, refers to an alkyl group, as defined herein, to which is attached at least one heterocycle substituent.

The term “(heterocycle)oxy,” as used herein, refers to a heterocycle, as defined herein, connected to the parent molecular group through an oxygen atom.

The term “(heterocycle)oxycarbonyl,” as used herein, refers to a (heterocycle)oxy group, as defined herein, connected to the parent molecular group through a carbonyl group.

The term “(heterocycle)sulfinyl,” as used herein, refers to a heterocycle group, as defined herein, connected to the parent molecular group through an —S(O)— group.

The term “(heterocycle)sulfonyl,” as used herein, refers to a heterocycle group, as defined herein, connected to the parent molecular group through an —SO₂— group.

The term “hydroxy,” as used herein, refers to —OH or a derivative thereof formed by replacement of the hydrogen atom thereon with a hydroxy protecting group.

The term “hydroxyalkyl,” as used herein, refers to an alkyl group, as defined herein, to which is attached at least one hydroxy substituent.

The term “hydroxyalkenyl,” as used herein, refers to an alkenyl group, as defined herein, to which is attached at least one hydroxy substituent, spaced apart from the carbon-carbon double bond by at least one sp carbon.

The term “hydroxy protecting group,” as used herein, refers to selectively introducible and removable groups which protect hydroxy groups against undesirable side reactions during synthetic procedures. Examples of hydroxy protecting groups include acetyl, formyl, chloroacetyl, trifluoroacetyl, methoxyacetyl, phenoxyacetyl, benzoyl, methyl, tert-butyl, 2,2,2-trichloroethyl, 2-trimethylsilylethyl, 1,1-dimethyl-2-propenyl, 3-methyl-3-butenyl, allyl, benzyl, para-methoxybenzyldiphenylmethyl, triphenylmethyl (trityl), tetrahydrofuryl methoxymethyl, methylthiomethyl, benzyloxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, and the like. Hydroxy protecting group can also be used as prodrug-forming groups.

The term “oxo,” as used herein, refers to a group formed by the replacement of two hydrogen atoms on the same carbon atom with a single oxygen atom.

The term “perfluoroalkoxy,” as used herein, refers to a perfluoroalkyl group attached to the parent group through an oxygen atom.

The term “perfluoroalkoxyalkyl,” as used herein, refers to an alkyl group, as defined herein, to which is attached at least one perfluoroalkoxy substituent.

The term “perfluoroalkoxyalkenyl,” as used herein, refers to an alkenyl group, as defined herein, to which is attached at least one perfluoroalkoxy substituent.

The term “perfluoroalkyl,” as used herein, refers to an alkyl group in which all of the hydrogen atoms have been replaced with fluorine atoms.

The term “thioalkanoyl, ” as used herein, refers to an alkyl group, as defined herein, connected to the parent molecular group through a thiocarbonyl.

The term “thioalkoxy,” as used herein, refers to an alkyl group, as defined herein, attached to the parent molecular group through a sulfur atom.

The term “thioalkoxyalkyl,” as used herein, refers to an alkyl group, as defined herein, to which is attached at least one thioalkoxy substituent.

The term “thioalkoxyalkenyl,” as used herein, refers to an alkenyl group, as defined herein, to which is attached at least one thioalkoxy substituent.

The term “thiocarbonyl,” as used herein, refers to —C(═S)—.

The term “thiocycloalkenyloxy,” as used herein, refers to a cycloalkenyl group, as defined herein, attached to the parent molecular group through a sulfur atom.

The compounds of the invention can exist as therapeutically acceptable salts. The term “therapeutically acceptable salt,” as used herein, refers to salts or zwitterionic forms of the compounds of the invention which are water or oil-soluble or dispersible, which are suitable for treatment of diseases without undue toxicity, irritation, and allergic response, which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, palmoate, pectinate, persulfate, 3-phenylpropionate, picrate, pivalate, propionate, succinate, tartrate, trichloroacetic, trifluoroacetic, phosphate, glutamate, bicarbonate, para-toluenesulfonate, and undecanoate. Also, amino groups in the compounds of the invention can be quaternized with as methyl, ethyl, propyl, and butyl chlorides, bromides and iodides; dimethyl, diethyl, dibutyl, and diamyl sulfates; decyl, lauryl, myristyl, and stearyl chlorides, bromides, and iodides; benzyl and phenethyl bromides. Examples of acids which can be employed to form therapeutically acceptable acid addition salts include inorganic acids such as hydrochloric, hydrobromic, sulfuric, and phosphoric and organic acids such as oxalic, maleic, succinic, and citric.

Basic addition salts can be prepared during the final isolation and purification of the compounds by reacting a carboxy group with a suitable base such as the hydroxide, carbonate, or bicarbonate of a metal cation or with ammonia or an organic primary, secondary or tertiary amine. Therapeutically acceptable salts cations based on lithium, sodium, potassium, calcium, magnesium, and aluminum and nontoxic quaternary ammonia and amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The term “sulfonyl,” as used herein, refers to —SO₂—.

In accordance with pharmaceutical compositions and methods of treatment, the compounds can be administered alone or in combination with other antibacterial, anti-psoriasis, anti-arthritis, and anti-chemotherapeutic toxicity agents. The therapeutically effective dose level depends on factors such as the disorder being treated and the severity of the disorder; the activity of the compound used; the composition employed; the age, body weight, general health, sex, and diet of the patient; the time of administration; the route of administration; the rate of excretion of the compound; the duration of treatment; and drugs used in combination with or coincidentally with the compounds. The compounds can be administered orally, parenterally, nasally, rectally, vaginally, or topically in unit dosage formulations containing therapeutically acceptable excipients such as carriers, adjuvants, diluents, vehicles, or combinations thereof. The term “parenteral” includes infusion, subcutaneous, intravenous, intramuscular, and intrasternal injection.

The antibacterial, anti-psoriasis, anti-arthritis, and anti-chemotherapeutic toxicity effect of parenterally administered compounds can be controlled by slowing their absorption, such as, for example, by administration of injectable suspensions of crystalline, amorphous, or otherwise water-insoluble forms of the compounds; administration of the compounds as oleaginous solutions or suspensions; or administration of microencapsulated matrices of the compounds trapped within liposomes, microemulsions, or biodegradable polymers. In each case, the ratio of compound to excipient and the nature of the excipient influences the rate of release of the compound. Transdermal patches also provide controlled delivery of compounds using rate-controlling membranes. Conversely, absorption enhancers can be used to increase absorption of the compounds.

Solid dosage forms for oral administration of the compounds include capsules, tablets, pills, powders, and granules. These compositions can contain diluents, lubricants, and buffering agents. Tablets and pills can be prepared with release-controlling coatings, and sprays can optionally contain propellants.

Liquid dosage forms for oral administration of the compounds include emulsions, microemulsions, solutions, suspensions, syrups, and elixirs. These compositions can also contain adjuvants such as wetting, emulsifying, suspending, sweetening, flavoring, and perfuming agents.

Topical dosage forms of the compounds include ointments, pastes, creams, lotions, gels, powders, solutions, sprays, and inhalants. Suppositories for rectal or vaginal administration comprise compounds with a suitable nonirritating excipient. Ophthalmic formulations such as eye drops and eye ointments are also contemplated as being within the scope of this invention.

The total daily dose of the compounds administered to a patient in single or divided doses can be in amounts from about 0.1 to about 200 mg/kg body weight or preferably from about 0.25 to about 100 mg/kg body weight. Single dose compositions contain these amounts or submultiples thereof to make up the daily dose.

### Determination of Antibacterial Activity

The minimum inhibitory concentrations (MIC's) of the compounds for the microorganisms listed in Table 1 were determined by the procedure described in National Committee for Clinical Laboratory Standards. 2000. Methods for Dilution Antimicrobial Susceptibility Tests for Bacteria That Grow Aerobically, 5th ed. Approved Standard: M7-A5 (NCCLS, Wayne, Pa.). Briefly, the compounds were dissolved in DMSO to 2 mg/mL and diluted in the appropriate susceptibility test medium to a concentration of 256 μg/mL. Serial two-fold dilutions were made in microtiter plates to achieve a final volume of 50 μL. Inocula for each organism were prepared by making a standard suspension in sterile saline with turbidity equivalent to that of a 0.5 McFarland Standard from an 18 to 24 hour culture grown on agar plates at 35° C. The standard suspension of each organism was diluted 100-fold in the appropriate medium and further diluted 2-fold by adding 50 μL to the medium containing antibiotic to achieve a final density of 5×10⁵ CFU/mL. Microdilution plates were incubated for 16 to 20 hours at 35° C. in ambient air. Each plate was visually inspected, and MIC's were recorded as the lowest concentration of drug which yielded no growth, a slight haze, or sparsely isolated colonies on the inoculum spot as compared to the growth control. The compounds inhibited the growth of these bacteria with MIC's in a range of about 0.125 μg/mL to about 128 μg/mL; in a more preferred range, the compounds inhibited the growth of bacteria with MIC's in a range of about 0.125 μg/mL to about 8 μg/mL; and in a most preferred range, the compounds inhibited the growth of bacteria with MIC's in a range of about 0.125 μg/mL to about 2 μg/mL.

Thus, the compounds are useful for treating bacterial infections such as, but not limited to, those shown below in Table 1.

TABLE 1
Microorganism

Staphylococcus aureus
NCTC 10649M
Staphylococcus epidermidis
3519
Moraxella catarrhalis
2604
Enterococcus faecium
ATCC GYR 1632
Streptococcus pneumoniae
ATCC 6303

### Preparation of the Compounds of the Invention

The compounds can be prepared by employing reactions shown in Schemes 1-7. It will be readily apparent to one of ordinary skill in the art that the compounds can be synthesized by substitution of the appropriate reactants in these syntheses, and that the steps themselves can be conducted in varying order. It will also be apparent that protection and deprotection steps can be performed to successfully complete the syntheses of the compounds. A thorough discussion of protecting groups is provided in Protective Groups in Organic Synthesis, 3rd edition, John Wiley & Sons, New York (1999). As used hereinbelow, R^{X} is hydrogen or alkyl; R^{Y} is aryl, heteroaryl, optionally substituted alkyl, optionally substituted cycloalkyl, or a nitrogen protecting group; and R^{Z} is aryl or heteroaryl.

Abbreviations used in the schemes and the examples are as follows: BINAP for 2,2′-bis(diphenylphosphino)-1,1′-binaphthyl; DME for dimethoxyethane; DMF for N,N-dimethylformamide; DMSO for dimethylsulfoxide; m-CPBA for meta-chloroperbenzoic acid; THF for tetrahydrofuran; PCC for pyridinium chlorochromate; PDC for pyridinium dichromate; Phth for phthalimide; DEAD for diethyl azodicarboxylate; DIAD for diisopropyl azodicarboxylate; AIBN for 2,2′-azobisisobutyronitrile; EDAC for 1-(3-dimethylaminopropyl)-3-ethylcarbodiimide; and Ac for acetyl.

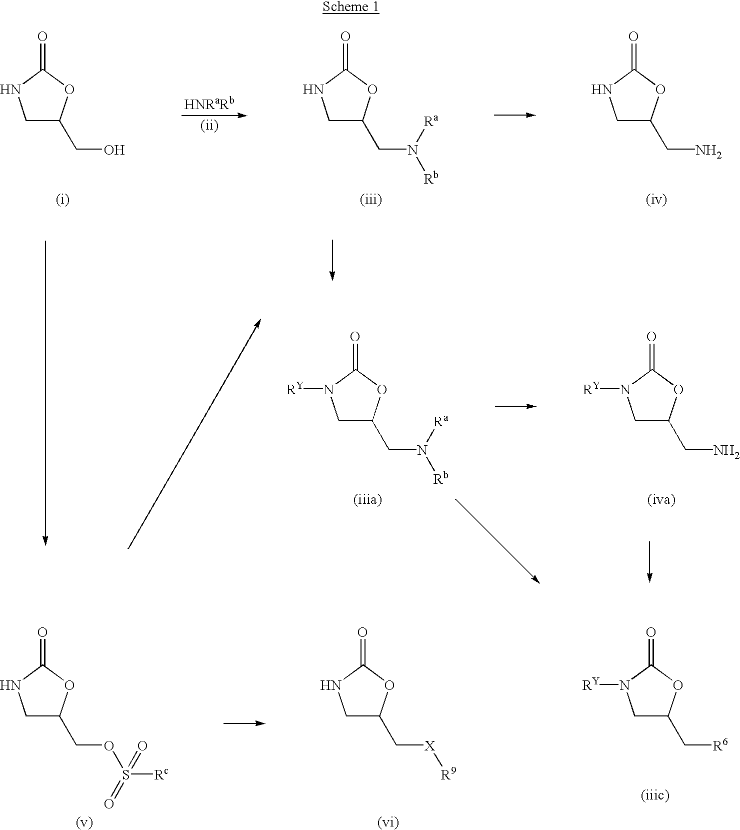

Conversion of 5-(hydroxymethyl)-1,3-oxazolidin-2-one (i) to compounds of formula (v) can be accomplished by treatment of the former with a hydroxyl activating group precursor such as para-toluenesulfonyl chloride (R^{c} is 4-methylphenyl), methanesulfonyl chloride (R^{c} is methyl), 2-nitrobenzenesulfonyl (R^{c} is 2-nitrophenyl), or trifluoromethanesulfonyl (R^{c} is trifluoromethyl) and a base such as diisopropylethylamine, pyridine, triethylamine, sodium carbonate, potassium carbonate, or cesium carbonate, followed by treatment of the compounds of formula (v) with the appropriate R⁶ introduction agent. For example, compounds of formula (vi) can be obtained by treatment of compounds of formula (v) with compounds of formula (M)⁺(XR⁹)⁻ wherein M is lithium, sodium or potassium, and X is O or S. The reactions are conducted at about 0° C. to about 30° C.; and the reaction times are from about 1 to about 24 hours. Solvents useful for this reaction include benzene, toluene, THF, dioxane, DME, or mixtures thereof.

Conversion of compounds of formula (vi), wherein X is S, to compounds wherein X is S(O) or SO₂ can be accomplished by treatment of the former with a oxidizing agent such as m-CPBA, potassium permanganate, or potassium peroxymonosulfate (Oxone®). The reactions are conducted at about 0° C. to about 30° C.; and the reaction times are from about 1 to about 10 hours, each depending on the degree of oxidation desired. Solvents useful for this reaction include benzene, toluene, THF, dioxane, dichloromethane, chloroform, DME, or mixtures thereof.

Conversion of compounds of formula (v) to compounds of formula (iii) can be accomplished by treatment of the former with an excess of the appropriately substituted amine wherein R^{a} and R^{b} are independently selected from hydrogen or unsubstituted or substituted alkyl, aryl, arylalkyl, cycloalkyl, cycloalkylalkyl, heteroaryl, and heteroarylalkyl. The reactions are conducted at about 20° C. to about 110° C.; and the reaction times are from about 1 to about 24 hours. Solvents useful for this reaction include the amines themselves, benzene, toluene, THF, dioxane, acetonitrile, DME, DMSO, or mixtures thereof. Compounds of formula (iii) in which either R^{a} or R^{b} is hydrogen can be further modified in order to introduce an alkanoyl group by treatment with an acylating agent in the presence of an appropriate base and in a solvent when required. Solvents useful for the reaction include pyridine, lutidine, dichloromethane, and mixtures thereof. Useful bases include pyridine, triethylamine, N,N-diisopropylethyamine, and the like. Acylating agent useful for the transformation include acetic anhydride, acetyl chloride, and the like. The temperature range is from about −10° C. to ambient temperature, and the reaction time ranges from about 2 hours to about 24 hours.

Conversion of compounds of formula (v) to compounds of formula (iii) can be accomplished by treatment of the former with compounds of formula (M)⁺(NR^{a}R^{b})⁻ wherein R^{a} and R^{b}, together with the nitrogen to which they are attached, are phthalimide and M is Na or K. The reactions are conducted at about 0° C. to about 100° C.; and the reaction times are from about 1 to about 24 hours. Solvents useful for this reaction include benzene, toluene, THF, dioxane, DME, DMF, or mixtures thereof.

Conversion of compounds of formula (i) to compounds of formula (iii) can also be accomplished by treatment of the former with phthalimide under Mitsunobu conditions (triphenylphosphine and DEAD or DIAD). The reactions are conducted at about −10° C. to about 30° C.; and the reaction times are from about 1 to about 10 hours. Solvents useful for this reaction include benzene, toluene, THF, dioxane, DME, dichloromethane, chloroform, or mixtures thereof.

Conversion of compounds of formula (iii) to compounds of formula (iiia) to introduce additional functionality appended to the oxazolidinone nitrogen are accomplished as described hereinbelow, wherein R^{c} is selected from aryl, heteroaryl, and a nitrogen protecting group.

Conversion of compounds of formula (iii), wherein R^{a} and R^{b}, together with the nitrogen to which they are attached, are phthalimide, to compounds of formula (iv) can be accomplished by treatment of the former with hydrazine. The reactions are conducted at about 50° C. to about 110° C.; and the reaction times are from about 1 to about 10 hours. Solvents useful for this reaction include benzene, toluene, THF, dioxane, DME, or mixtures thereof. Likewise, such transformations convert compounds of formula (iiia) to compounds of formula (iva).

Conversion of compounds of formula (iiia) and (iva) to compounds of formula (iiic) can be achieved by one skilled in the art to remove protecting groups or introduce functionalities such as, but not limited to, amides, carbamates, ureas, ethers, thioethers, sulfoxides, and sulfones.

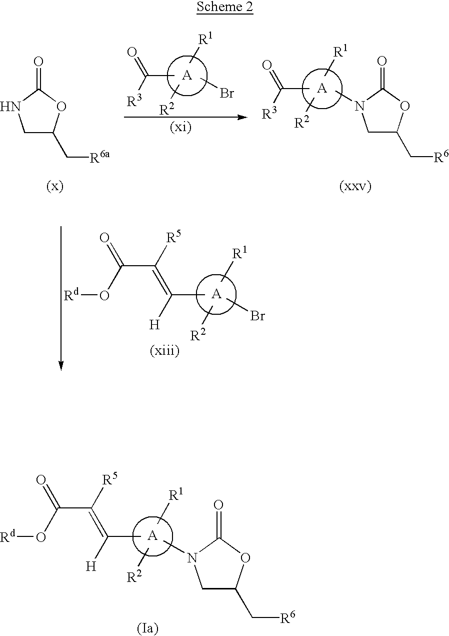

Conversion of compounds of formula (x), wherein R^{6a} is N-Phth or a doubly protected primary amine , that is NR⁷R⁸, in which such protecting groups as acetyl and 2,4-dimethoxybenzyl are simultaneously used, to compounds of formula (xxv) can be accomplished by treatment of the former with compounds of formula (xi), a palladium catalyst such as tris(dibenzylideneacetone)dipalladium, palladium(II) acetate, bis(triphenylphosphine)palladium(II) chloride, or tetrakis(triphenylphosphine)palladium, and, optionally, an additive such as tributylphosphine, triphenylphosphine, triphenylarsine, (2-(diphenylphosphino)ethyl)(diphenyl)phosphine, (3-(diphenylphosphino)propyl)(diphenyl)phosphine, tri-tert-butylphosphine or BINAP, and a base such as sodium carbonate, potassium carbonate, or cesium carbonate. The reactions are conducted at about 50° C. to about 110° C.; and the reaction times are from about 1 to about 24 hours. Solvents useful for this reaction include benzene, toluene, THF, dioxane, DME, or mixtures thereof.

Conversion of compounds of formula (x) to compounds of formula (Ia) (R^{d} is alkyl, preferably methyl or ethyl) can be accomplished by treatment of the former with compounds of formula (xiii) using the same reagents and conditions described for the conversion of compounds of formula (x) to compounds of formula (xxv).

The syntheses of compounds of formulas (xxii) and (xiii) is shown in Scheme 3.

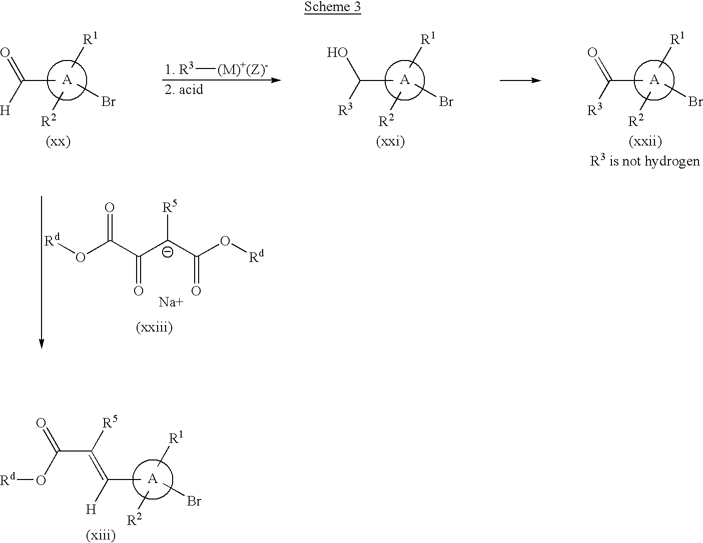

Conversion of compounds of formula (xx) to compounds of formula (xxi) can be accomplished by treatment of the former with compounds of formula R³-(M)⁺(Z)⁻ wherein R³, M and Z combine to form organometallic reagents such as, for example, organomagnesium halides, organozinc halides, organocopper iodides, or organocadmium halide reagents. The reactions are conducted at about 0° C. to about 100° C.; and the reaction times are from about 1 to about 24 hours. Solvents useful for this reaction include benzene, toluene, THF, dioxane, diethyl ether, DME, or mixtures thereof.

Conversion of compounds of formula (xxi) to compounds of formula (xxii) can be accomplished by treatment of the former with oxidizing agents such as CrO₃/H₂SO₄, PDC, PCC, oxalyl chloride/DMSO, or SO₃.pyridine/DMSO. The reactions are conducted at about −15° C. to about 25° C.; and the reaction times are from about 1 to about 24 hours, each depending on the reaction chosen.

Conversion of compounds of formula (xx) to compounds of formula (xiii) can be accomplished by treatment of the former with compounds of formula (xxiii). The reactions are conducted at about 0° C. to about 110° C.; and the reaction times are from about 1 to about 24 hours.

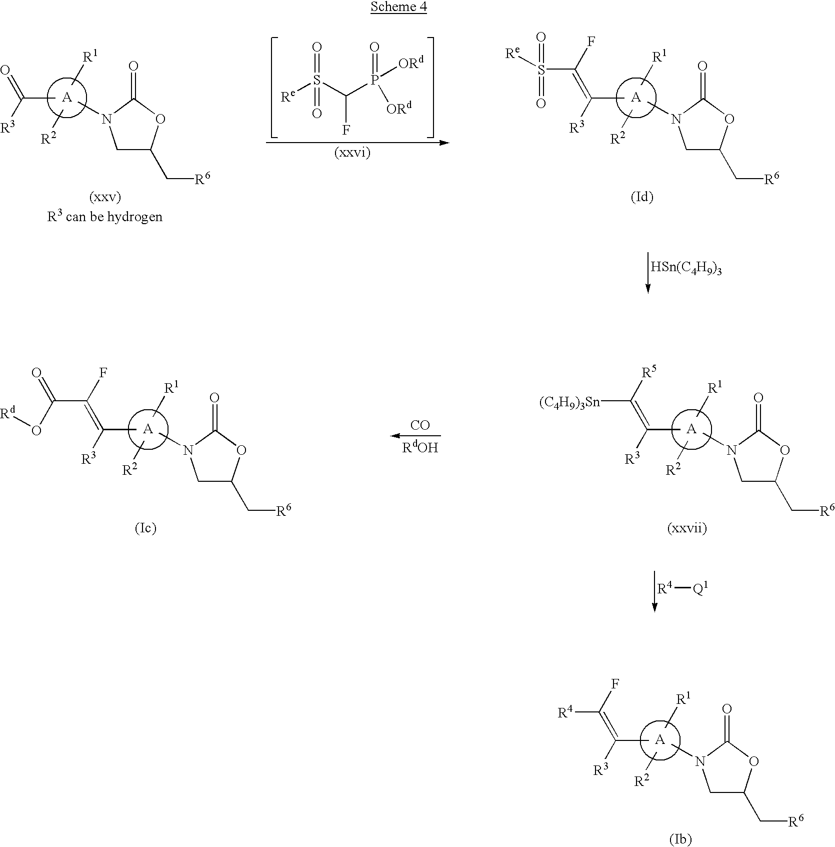

Conversion of compounds of formula (xxv) to compounds of formula (Id) can be accomplished by treatment of the former with compounds of formula (xxvi), prepared in situ as described hereinbelow (R^{e} is optionally substituted alkyl, optionally substituted cycloalkyl, optionally substituted aryl, or optionally substituted heteroaryl) and a base such as sodium carbonate, potassium carbonate, sodium hydride, potassium bis(trimethylsilyl)amide, or lithium bis(trimethylsilyl)amide. The reactions are conducted at about 0° C. to about 110° C.; and the reaction times are from about 1 to about 24 hours. Solvents useful for this reaction include benzene, toluene, THF, dioxane, DME, or mixtures thereof.

Conversion of compounds of formula (Id) to compounds of formula (xxvii) can be accomplished by treatment of the former with tributyltin hydride and a free radical initiator such as AIBN. These reactions are conducted at about 0° C. to about 25° C.; and the reaction times are about 1 to about 24 hours. Solvents useful for this reaction include benzene, toluene, THF, dioxane, DME, or mixtures thereof.

Conversion of compounds of formula (xxvii) to compounds of formula (Ib) can be accomplished by treatment of the former with compounds of formula R⁴-Q¹ (R⁴ is optionally substituted alkenyl, optionally substituted cycloalkenyl, optionally substituted aryl ,or optionally substituted heteroaryl, and Q¹ is bromide, iodide, or trifluoromethanesulfonate), a palladium catalyst such as tris(dibenzylideneacetone)dipalladium, palladium(II) acetate, bis(triphenylphosphine)palladium(II) chloride, or tetrakis(triphenylphosphine)palladium, and, optionally, an additive such as tributylphosphine, triphenylphosphine, triphenylarsine, (2-(diphenylphosphino)ethyl)(diphenyl)phosphine, (3-(diphenylphosphino)propyl)(diphenyl)phosphine, tritert-butylphosphine or BINAP, and optionally a salt such as copper(I) bromide or copper(I) iodide. The reactions are conducted at about 50° C. to about 110° C.; and the reaction times are from about 1 to about 48 hours. Solvents useful for this reaction include DMF, toluene, THF, dioxane, DME, or mixtures thereof.

Conversion of compounds of formula (xxvii) to compounds of formula (Ic) can be accomplished by replacing the compounds of formula R⁴-Q¹ with carbon monoxide gas and conducting the reaction in an alcohol solvent such as methanol or ethanol. The reactions are typically conducted at about 0° C. to about 100° C.; and the reaction times are typically from about 1 to about 24 hours.

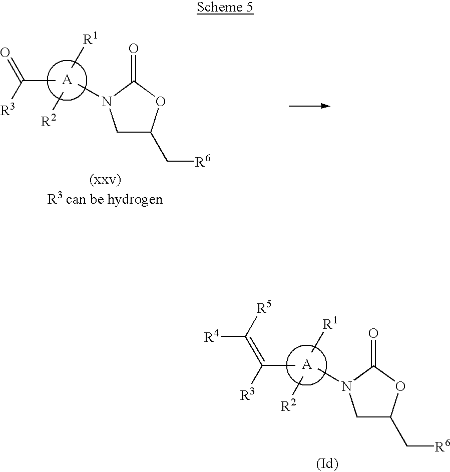

Conversion of compounds of formula (xxv) to compounds of formula (Id) can be accomplished by treatment of the former with methyl(triphenyl)phosphonium chloride, methyl(triphenyl)phosphonium bromide, methyl(triphenyl)phosphonium iodide, (4-cyanobenzyl)triphenylphosphonium chloride and the like, or a dialkylmethylphosphonate and a base such as sodium hydride, potassium hydride, lithium bis(trimethylsilyl)amide, sodium bis(trimethylsilyl)amide, or potassium bis(trimethylsilyl)amide. The reactions are conducted at about 0° C. to about 100° C.; and the reaction times are from about 1 to about 24 hours. Solvents useful for this reaction include benzene, toluene, THF, dioxane, DMSO, DME, or mixtures thereof.

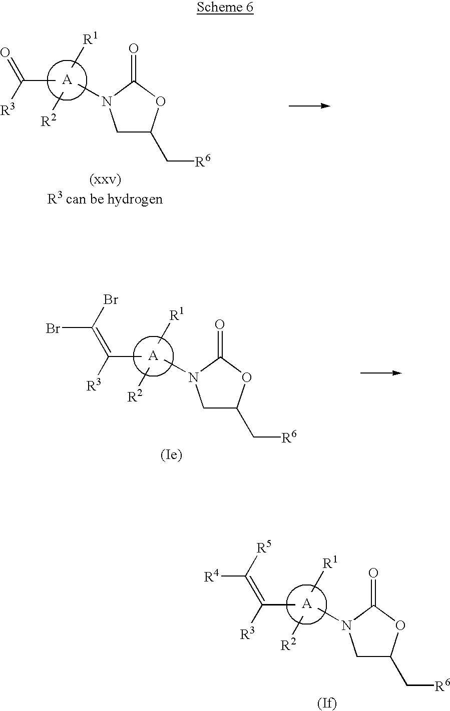

As shown in Scheme 6, conversion of compounds of formula (xxv) to compounds of formula (Ie) can be accomplished by treatment of the former with carbon tetrabromide and triphenylphosphine or triphenylphosphine supported on cross-linked polystyrene. The reactions are typically conducted at about −15° C. to about 0° C.; and the reaction times are typically from about 1 to about 5 hours. Solvents useful for this reaction include dichloromethane, and chloroform.

Conversion of compounds of formula (Ie) to compounds of formula (If) can be accomplished by treatment of the former with compounds of formula M¹-R⁴ (M¹ is trialkyl or triarylstannyl, boronic acid or ester, lithium, zinc, or zirconium; and R⁴ is unsubstituted or substituted aryl, heteroaryl, alkenyl, or cycloalkenyl), a palladium catalyst such as tris(dibenzylideneacetone)dipalladium, bis(triphenylphosphine)palladium(II) chloride, palladium(II)acetate or tetrakis(triphenylphosphine)palladium, and, optionally, an additive such as tributylphosphine, triphenylphosphine, triphenylarsine, tri-t-butylphosphine, tri-2-furylphosphine (2-(diphenylphosphino)ethyl)(diphenyl)phosphine, (3-(diphenylphosphino)propyl)(diphenyl)phosphine, or BINAP, and a base as sodium carbonate, potassium carbonate, or cesium carbonate. The reactions are conducted at about 50° C. to about 110° C.; and the reaction times are from about 1 to about 48 hours. Solvents useful for this reaction include benzene, toluene, THF, dioxane, DME, or mixtures thereof.

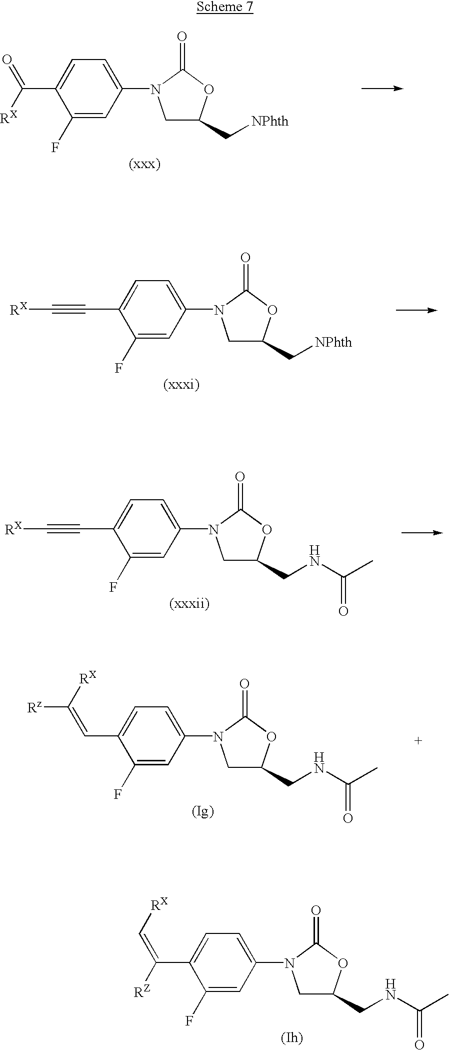

Compounds of formula (xxx), wherein, R^{X} can be hydrogen or alkyl, can be converted to compounds of formula (xxxi) by treatment of the former with dimethyl diazomethylphosphonate and a base such as potassium tert-butoxide in a solvent such as THF at −78° C. to 20° over approximately 24 hours. Treatment of compounds of formual (xxxi) with hydrazine as previously described removes the phthalimide. The revealed primary amine may then be acetylated under standard conditions elaborated hereinabove to furnish compounds of formula (xxxii). Conversion of compounds of formula (xxxii) to compounds of formula (Ig) and (Ih) can be accomplished by treatment with an appropriate borane such as diisoamylborane in a solvent such as THF at a temperature of −30° to 0° C. for about 1 hour. This is followed by treatment with an aryl bromide or heteroaryl bromide along with an additive such as tetrakis(triphenylphosphine)palladium and a base such as potassium carbonate. The reaction is heated to between 50° C. and 100° C. for approximately 24 hours.

In addition to the chemistry discussed above, other standard manipulations can be used to prepare compounds of the invention. Amino groups can be reacted with acid chlorides, acid anhydrides, isocyanates, chloroformates, and aldehydes under reductive alkylation conditions (sodium borohydride, sodium cyanoborohydride) to provide variety of substituent groups. Carboxamido groups can be reacted with Lawesson's reagent or P₄S₁₀ Na₂CO₃ to provide thioamides.

Heteroaryl substituents such as furan or thiazole can be converted to carboxylic acid by hydrolytic or oxidative means well-known in the art. Once formed, the carboxylic acid groups can be converted to alkoxycarbonyl, carboxamide, or cyano groups by esterification, coupling, and dehydration procesures.

A thorough discussion of reactions which are useful for preparing the compounds are described in Larock, Comprehensive Organic Transformations. A Guide to Functional Group Preparations, John Wiley & Sons (1999).

The invention will now be described in connenction with preferred embodiments of Schemes 1-7, which are not intended to limit its scope. On the contrary, the invention covers all alternatives, modifications, and equivalents which are included within the scope of the claims. It will also be apparent that protection and deprotection steps can be performed to successfully complete the syntheses of the compounds. Thus, the following examples show an especially preferred practice of the invention, it being understood that the examples are for the purposes of illustration of certain preferred embodiments and are presented to provide what is believed to be the most useful and readily understood description of its procedures and conceptual aspects.

### EXAMPLE 1

### N-(((5S)-3-(4-((Z)-2-(5-acetyl-2-thienyl)-2-bromoethenyl)-3-fluorophenyl)-2-oxo-1,3-oxazolidin-5-yl)methyl)acetamide

### EXAMPLE 1A

### ((5R)-2-oxo-1,3-oxazolidin-5-yl)methyl 4methylbenzenesulfonate

A solution of (5R)-5-(hydroxymethyl)-1,3-oxazolidin-2-one (10.0 g), prepared as described in Tetrahedron: Asymmetry 1995, 6, 1181-1190, in pyridine (60 mL) at −10° C. was treated with para-toluenesulfonyl chloride (21.2 g), stirred for 2 hours, poured into 1:1 brine/water (100 mL), and extracted with ethyl acetate. The extract was washed sequentially with 1:1 saturated sodium bicarbonate/water and 1:1 brine/water, and the aqueous washes were extracted with ethyl acetate. The extract was dried (MgSO₄), filtered, and concentrated to provide the desired product. MS (ESI(+)) m/e 272 (M+H)⁺; ¹H NMR (300 MHz, CDCl₃) δ7.80 (d, 2H), 7.39 (d, 2H), 4.80 (m, 1H), 4.18 (d, 2H), 3.70 (t, 1H), 3.50 (dd, 1H), 2.45 (s, 3H).

### EXAMPLE 1B

### 2-(((5R)-2-oxo-1,3-oxazolidin-5-yl)methyl)1-isoindole-1,3(2)-dione

A solution of Example 1A (22.16 g) in DMF (163 mL) was treated with potassium phthalimide (16.7 g), heated to 80° C., stirred for 5 hours, poured into 1 :1 brine/water (200 mL), and extracted with dichloromethane. The extract was dried (MgSO₄), filtered, and concentrated. The concentrate was treated with ethyl acetate (200 mL), cooled to 5° C., and filtered. The mother liquor was concentrated, treated with ethyl acetate (100 mL), cooled to 5° C., filtered, and combined with the first crop to provide the desired product. MS (ESI(+)) m/e 247 (M+H)⁺; ¹H NMR (300 MHz, CDCl₃) δ7.88 (m, 2H), 7.75 (m, 2H), 4.98 (m, 1H), 4.10 (dd, 1H), 3.90 (dd, 1H), 3.73 (t, 1H), 3.49 (dd, 1H).

### EXAMPLE 1C

### 4-((5S)-5-((1,3-dioxo-1,3-dihydro-2H-isoindol-2-yl)methyl)-2-oxo-1,3-oxazolidin-3-yl)2-fluorobenzaldehyde

A suspension of Example 1B (2.5 g) in toluene (20 mL) in a sealable tube was deoxygenated with nitrogen and treated sequentially with 4-bromo-2-fluorobenzaldehyde (2.03 g), BINAP (498 mg), cesium carbonate (4.56 g), and tris(dibenzylideneacetone)dipalladium (366 mg). The tube was sealed, and the mixture was heated at 100° C. for 24 hours, cooled, poured into 1:1 saturated ammonium chloride/water (200 mL), and extracted with ethyl acetate. The extract was dried (MgSO₄), filtered, and concentrated. The concentrate was treated with dichloromethane (100 mL), cooled to 5° C., and filtered. The mother liquor was concentrated, treated with dichloromethane (70 mL), cooled to 5° C., filtered, and combined with the first crop to provide the desired product. mp 167-169° C.; MS (ESI(+)) m/e 369 (M+H)⁺; ¹H NMR (300 MHz, CDCl₃) δ10.27 (s, 1H), 7.88 (m, 3H), 7.75 (m, 2H), 7.63 (dd, 1H), 7.27 (dd, 1H), 5.04 (m, 1H), 4.16 (m, 2H), 4.00 (m, 2H).

### EXAMPLE 1D

### (((5S)-3-(4-(2,2dibromovinyl)-3-fluorophenyl)-2-oxo-1,3-oxazolidin-5-yl)methyl)-1H-isoindole-1,3(2H)-dione

Polymer-supported triphenylphosphine (10.0 g of 3 mmol/g, 2% divinylbenzene cross-linked polystyrene resin) was swelled in dichloromethane (100 mL) and cooled to −10° C., treated with carbon tetrabromide (4.97 g), warmed to −5° C., stirred for 30 minutes, treated portionwise with Example 1C (1.84 g), stirred for 20 minutes, treated with methanol (50 mL) and filtered. The resin was washed sequentially with dichloromethane (100 mL), 1:1 dichloromethane/methanol (100 mL), and methanol (3×100 mL). The washings were combined, and the solution was concentrated. The concentrate was dissolved in dichloromethane (150 mL) and washed with 1:1 saturated sodium bicarbonate/water and 1:1 brine/water. These washes were extracted with dichloromethane. The extract was dried (MgSO₄), filtered, and concentrated to provide the desired product. MS (ESI(+)) m/e 542 (M+NH₄)⁺; ¹H NMR (300 MHz, CDCl₃) δ7.89 (m, 2H), 7.77 (m, 3H), 7.50 (s, 1H), 7.46 (dd, 1H), 7.23 (dd, 1H), 5.01 (m, 1H), 4.16 (m, 2H), 3.98 (m, 2H).

### EXAMPLE 1E

### N-(((5S)-3-(4-(2,2-dibromovinyl)-3-fluorophenyl)-2-oxo-1,3-oxazolidin-5-yl)methyl)acetamide

A suspension of Example 1D (2.45 g) in 1:1 THF/ethanol (36 mL) was heated to 70° C., treated with hydrazine monohydrate (3×227 mL) at 1 hour intervals, cooled to 25° C., and filtered. The filtrate was concentrated, dissolved in a mixture of pyridine (10 mL) and dichloromethane (25 mL), cooled to −5° C., and treated with acetic anhydride (880 μL), warmed to room temperature, stirred for 5 minutes, and concentrated to provide the desired product. mp 177-179° C.; MS (ESI(+)) m/e 437 (M+H)⁺; ¹H NMR (300 MHz, CDCl₃) δ7.81 (t, 1H), 7.50 (s, 1H), 7.47 (dd, 1H), 7.20 (dd, 1H), 5.92 (br t, 1H), 4.80 (m, 1H), 4.06 (t, 1H), 3.77 (dd, 1H), 3.75-3.55 (m, 2H), 2.02 (s, 3H).

### EXAMPLE 1F

### N-(((5S)-3-(4-((Z)-2-(5-acetyl-2-thienyl)-2-bromoethenyl)-3-fluorophenyl)-2-oxo-1,3-oxazolidin-5-yl)methyl)acetamide

A mixture of Example 1E (80 mg), 5-acetyl-2-thienylboronic acid (37.4 mg), and tris(dibenzylideneacetone)dipalladium (11 mg) in deoxygenated dimethoxyethane (2 mL) was treated with 2N sodium carbonate (0.34 mL), heated to 55° C. for 20 hours, and concentrated. The concentrate was dissolved in dichloromethane (2 mL), filtered, and concentrated. The concentrate was purified by flash column chromatography on silica gel with 1:1 hexanes/acetone to provide the desired product. mp 187-190° C. (dec.); MS (ESI(+)) m/e 482 (M+H)⁺; ¹H NMR (300 MHz, CDCl₃) δ8.07 (t, 1H), 7.61 (d, 1H), 7.55 (dd, 1H), 7.53 (s, 1H), 7.40 (d, 1H), 7.23 (dd, 1H), 5.95 (br t, 1H), 4.80 (m, 1H), 4.08 (t, 1H), 3.80 (dd, 1H), 3.68 (m, 2H), 2.51 (s, 3H), 2.02 (s, 3H).

### EXAMPLE 2

### N-(((5S)-3-(4-((Z)-2-bromo-2-(3pyridinyl)ethenyl)-3-fluorophenyl)-2-oxo-1,3-oxazolidin-5-yl)methyl)acetamide

The desired product was prepared by substituting 3-pyridinylboronic acid for 5-acetyl-2-thienylboronic acid in Example IF. MS (ESI(+)) m/e 435 (M+H)⁺; ¹H NMR (300 MHz, CDCl₃) δ7.81 (t, 1H), 7.5 (s, 1H), 7.47 (dd, 1H), 7.2 (dd, 1H), 5.92 (br t, 1H), 4.8 (m, 1H), 4.06 (t, 1H), 3.77 (dd, 1H), 3.18 (m, 2H), 2.02 (s, 3H).

### EXAMPLE 3

### N-(((5S)-3-(4-((Z)-2-bromo-2-(2-furyl)ethenyl)-3-fluorophenyl)-2-oxo-1,3-oxazolidin-5-yl)methyl)acetamide

The desired product was prepared by substituting 2-furylboronic acid for 5-acetyl-2-thienylboronic acid in Example 1F. mp 176-178° C. (dec.); MS (ESI(+)) m/e 424 (M+H)⁺; ¹H NMR (300 MHz, CDCl₃) δ8.06 (t, 1H), 7.56 (s, 1H), 7.51 (dd, 1H), 7.47 (s, 1H), 7.20 (dd, 1H), 6.70 (d, 1H), 6.45 (q, 1H), 5.96 (br t, 1H), 4.80 (m, 1H), 4.08 (t, 1H), 3.77 (dd, 1H), 3.75-3.55 (m, 2H), 2.03 (s, 3H).

### EXAMPLE 4

### N-(((5S)-3-(4-((Z)-2bromo-2-(3,5-dimethyl-4-isoxazolyl)ethenyl)-3-fluorophenyl)-2-oxo-1,3-oxazolidin-5-yl)methyl)acetamide

The desired product was prepared by substituting 3,5-dimethyl-4-isoxazolylboronic acid for 5-acetyl-2-thienylboronic acid in Example 1F. MS (ESI(+)) m/e 453 (M+H)⁺; ¹H NMR (300 MHz, CDCl₃) δ8.01 (t, 1H), 7.55 (dd, 1H), 7.24 (dd, 1H), 6.90 (s, 1H), 5.92 (br t, 1H), 4.80 (m, 1H), 4.07 (t, 1H), 3.81 (dd, 1H), 3.75-3.55 (m, 2H), 2.48 (s, 3H), 2.36 (s, 3H), 2.03 (s, 3H).

### EXAMPLE 5

### N-(((5S)-3-(4-((Z)-2-(4-cyanophenyl)ethenyl)phenyl-2-oxo- 1,3-oxazolidin-5-yl)methyl)acetamide

A suspension of N-(((5S)-3-(4-formylphenyl)-2-oxo-1,3-oxazolidin-5-yl)methyl)acetamide (0.08 g), prepared as described in J. Med. Chem. 1990, 33, 2569-2578, in dichloromethane (4 mL) at room temperature was treated sequentially with (4-cyanobenzyl)(triphenyl)phosphonium chloride (0.28 g) and 0.6M lithium bis(trimethylsilyl)amide in toluene (1.1 mL), stirred for 20 hours, treated with water, and extracted with dichloromethane. The extract was washed with brine (10 mL), dried (MgSO₄), filtered, and concentrated. The concentrate was purified by flash column chromatography on silica gel with 2:1 hexanes/acetone to 1:1 hexanes/acetone to provide the desired product as a 2:1 mixture of Z/E isomers. MS (APCI(+)) m/e 362 (M+H)⁺; ¹H NMR (300 MHz, DMSO-d₆) δ7.68-7.50 (m, 4H), 7.40 (d, 1H), 7.31(d, 1H), 7.20 (d, 2H), 7.22-7.02 (dd, 1H), 6.76-6.52 (dd, 1H), 6.27 (m, 1H), 4.76 (m, 1H), 4.15-4.08 (m, 1H), 3.78-3.58 (m, 3H), 2.02 (s, 3H).

### EXAMPLE 6

### ethyl (2Z)-3-(4-((5S)-5-((acetylamino)amino)methyl)-2-oxo-1,3-oxazolidin-3-yl)phenyl)-2-fluoro-2-propenoate

### EXAMPLE 6A

### ethyl (2Z)-3-(4-bromophenyl)-2-fluoro-2-propenoate

A suspension of the sodium enolate of 2-fluoro-3-oxo-succinic acid diethyl ester (2.0 g), prepared as described in J. Chem. Soc. 1955, 2190-2193, in THF (12 mL) at 0° C. was treated with 4-bromobenzaldehyde (811 mg), stirred for 2 hours, heated to reflux, stirred for 4 hours, and concentrated. The concentrate was partitioned between diethyl ether (50 mL) and saturated sodium bicarbonate (50 mL) and stirred for 45 minutes. The aqueous phase was extracted with diethyl ether, and the extract was dried (Na₂SO₄), filtered, and concentrated. The concentrate was purified by flash column chromatography on silica gel with 98:2 hexanes/ethyl acetate to provide the desired product. mp 59-61° C.; MS (DCI/NH₃) m/e 290 (M+NH₄)⁺; ¹H NMR (300 MHz, CDCl₃) δ7.54 (d, 2H), 7.50 (d, 2H), 6.85 (d, 1H), 4.35 (q, 2H), 1.39 (t, 3H).

### EXAMPLE 6B

### ((5R)-2-oxo-1,3-oxazolidin-5-yl)methyl2-nitrobenzenesulfonate

A solution of (5R)-5-(hydroxymethyl)-1,3-oxazolidin-2-one, prepared as described in Tetrahedron: Asymmetry 1995, 6, 1181-1190, (1.2 g) in pyridine (5 mL) at −10° C. was treated with a solution of 2-nitrobenzensulfonyl chloride (2.75 g) in pyridine (3 mL), warmed to −5° C., stirred for 2.5 hours, treated with water, and extracted with ethyl acetate. The extract was washed with saturated sodium bicarbonate and brine, dried (MgSO₄), filtered, and concentrated. The concentrate was triturated with 1:3 acetone/hexanes to provide the desired product. MS (APCI(+)) m/e 303 (M+H)⁺; ¹H NMR (300 MHz, DMSO-d₆) δ8.15 (m, 2H), 8.04 (t, 1H), 7.96 (t, 1H), 7.60 (br s, 1H), 4.83 (m, 1H), 4.48-4.34 (m, 2H), 3.57-3.51 (t, 1H), 3.22 (dd, 1H).

### EXAMPLE 6C

### N-(2,4-dimethoxybenzyl)-N-(((5R)-2-oxo-1,3-oxazolidin-5-yl)methyl)acetamide

A solution of Example 6B (11.75 g) in DMSO (50 mL) at room temperature was treated with 2,4-dimethoxybenzylamine (7 mL), stirred for 16 hours, treated with water (30 mL), and extracted with 1:1 ethyl acetate/diethyl ether. The extract was washed with brine, dried (MgSO₄), filtered, and concentrated. The concentrate was dissolved in dichloromethane (50 mL), treated with pyridine (6.5 mL) and acetic anhydride (3.7 mL), stirred for 20 hours, and concentrated. The concentrate was dissolved in ethyl acetate, washed with water and brine, dried (MgSO₄), filtered, and concentrated. The concentrate was purified by flash column chromatography on silica gel with 70:30 hexanes/acetone to 70:30 acetone/hexanes to provide the desired product. MS (APCI(+)) m/e 309 (M+H)⁺; ¹H NMR (500 MHz, DMSO-d₆) (rotamers) δ7.56 and 7.46 (br s, 1H), 6.95 (d, 1H), 6.60 and 6.54 (s, 1H), 6.53 and 6.47 (dd, 1H), 4.76 and 4.56 (m, 1H), 4.51 and 4.42 (m, 2H), 3.79 and 3.75 (s, 6H), 3.35 and 3.30 (m, 1H), 3.62 and 3.59 (m, 1H), 3.11 and 3.06 (m, 1H), 3.50 and 3.45 (m, 1H), 2.09 and 2.05 (s, 3H); ¹³C NMR (125 MHz, DMSO-d₆) (rotamers) δ170.7 and 170.0, 160.1 and 159.6, 158.4 and 158.3, 157.9 and 157.8, 128.6 and 128.2, 117.3 and 116.9, 104.5, 98.6 and 98.2, 74.0 and 74.1, 55.3, 55.2, 51.1 and 47.8, 47.6 and 42.6, 42.8 and 42.6, 21.4 and 21.2.

### EXAMPLE 6D

### ethyl (2Z)-3-(4((5S)-5-((acetylamino)methyl)-2-oxo-1,3-oxazolidin-3-yl)phenyl)-2-fluoro-2-propenoate

A sealable tube containing Cs₂CO₃ (163 mg) was treated sequentially with S-2,2′-bis(diphenylphosphino)-1,1′-binaphthyl (11 mg), tris(dibenzylideneacetone)dipalladium (10 mg), Example 6C (110 mg), toluene (5 mL), and Example 6A (117 mg). After the addition of each reactant, the vessel was evacuated and filled with nitrogen. The tube was sealed, and the mixture was heated to 100° C. for 20 hours, cooled to room temperature, treated with saturated ammonium chloride (40 mL), and extracted with ethyl acetate. The extract was dried (Na₂SO₄), filtered, and concentrated. The concentrate was dissolved in dichloromethane (10 mL), treated with trifluoroacetic acid (10 mL), stirred for 2 hours, treated with methanol, and concentrated. The concentrate was dissolved in ethyl acetate (40 mL), washed with saturated sodium bicarbonate, dried (Na₂SO₄), filtered, and concentrated. The concentrate was purified by flash column chromatography on silica gel with 80:20 dichloromethane/acetone to provide the desired product. mp 161-163° C.; MS (DCI/NH₃) m/e 368 (M+NH₄)⁺; ¹H NMR (300 MHz, DMSO-d₆) δ8.23 (t, 1H), 7.75 (d, 2H), 7.63, (d, 2H), 7.05 (d, 1H), 4.64 (m, 1H), 4.29 (q, 2H), 4.15 (t, 1H), 3.77 (dd, 1H), 3.43 (t, 2H), 1.83 (s, 3H), 1.30 (t, 3H).

### EXAMPLE 7

### N-(((5S)-3-(4-((Z)-2-bromo-2-(3-cyanophenyl)ethenyl)-3fluorophenyl)-2-oxo-1,3-oxazolidin-5-yl)methyl)acetamide

The title compound was prepared according to the method described in Example 1F, substituting 3-cyanophenylboronic acid for 5-acetylthiophene-2-boronic acid to afford the desired product. MS (ESI): m/e 459 (M+1)⁺; ¹H NMR (300 MHz, CDCl₃) δ8.04 (t, 1H), 7.95 (s, 1H), 7.87 (d, 1H), 7.62 (t, 1H), 7.56 (dd, 1H), 7.5 (d, 1H), 7.3 (s, 1H), 7.25 (dd, 1H), 6.02 (bt, 1H), 4.82 (m, 1H), 4.1 (t, 1H), 3.82 (dd, 1H), 3.6-3.75 (m, 2H), 2.04 (s, 3H).

### EXAMPLE 8

### N-(((5S)-3-(4-((Z)-2-bromo-2-(3-formylphenyl)vinyl)-3-fluorophenyl)-2-oxo-1,3-oxazolidin-5-yl)methyl)acetamide

The title compound was prepared according to the method described in Example 1F, substituting 3-formylphenylboronic acid for 5-acetylthiophene-2-boronic acid to afford the desired product.

MS (ESI): m/e 462 (M+1)^{+;} ¹H NMR (300 MHz, CDCl₃) δ10.1 (s, 1H), 8.15 (s, 1H), 8.05 (t, 1H), 7.8-8.0 (m, 2H), 7.5-7.7 (m, 2H), 7.35 (s, 1H), 7.2 (dd, 1H), 6.45 (bt, 1H), 4.8 (m, 1H), 4.1 (t, 1H), 3.8-3.85 (dd, 1H), 3.6-3.75 (m, 2H), 2.05 (s, 3H).

### EXAMPLE 9

### N-(((5S )-3-(4-((Z)-2-bromo-2-(3-(hydroxymethyl)phenyl)ethenyl)-3-fluorophenyl)-2-oxo-1,3-oxazolidin-5-yl)methyl)acetamide

The title compound was prepared according to the method described in Example 1F, substituting 3-(hydroxymethyl)phenylboronic acid for 5-acetylthiophene-2-boronic acid to afford the desired product. MS (ESI): m/e 464 (M+1)⁺; ¹H NMR (300 MHz, CDCl₃) δ8.03 (t, 1H), 7.68 (s, 1H), 7.58 (m, 1H), 7.52 (dd, 1H), 7.3-7.4 (m, 3H), 7.23 (dd, 1H), 4.8 (m, 1H), 4.7 (s, 2H), 4.1 (t, 1H), 3.83 (dd, 1H), 3.5-3.7 (m, 2H), 2.01 (s, 3H).

### EXAMPLE 10

### N-(((5S)-3-4-((Z)-2-bromo-2-(4-(hydroxymethyl)phenyl)ethenyl)-3-fluorophenyl)-2-oxo-1,3-oxazolidin-5-yl)methyl)acetamide

The title compound was prepared according to the method described in Example 1F, substituting 4-(hydroxymethyl)phenylboronic acid for 5-acetylthiophene-2-boronic acid to afford the desired product. MS (ESI): m/e 464 (M+1)⁺; ¹H NMR (300 MHz, CDCl₃) δ8.04 (t, 1H), 7.66 (d, 2H), 7.52 (d, 1H), 7.38 (d, 2H), 7.25 (s, 1H), 7.21 (dd, 1H), 5.97 (bt, 1H), 4.8 (m, 1H), 4.74 (s, 2H), 4.09 (t, 1H), 3.81 (dd, 1H), 3.6-3.8 (m, 2H), 2.03 (s, 3H).

### EXAMPLE 11

### N-(((5S)-3-(4-((Z)-2-(3-acetylphenyl)-2-bromoethenyl)-3-fluorophenyl)2-oxo-1,3-oxazolidin-5-yl)methyl)acetamide

The title compound was prepared according to the method described in Example 1F, substituting 3-acetylphenylboronic acid for 5-acetylthiophene-2-boronic acid to afford the desired product. MS (ESI): m/e 476 (M+1)⁺; ¹H NMR (300 MHz, CDCl₃) δ8.22 (t, 1H), 8.04 (t, 1H), 7.92 (d, 1H), 7.87 (d, 1H), 7.55 (dd, 1H), 7.5 (d, 1H), 7.3 (s, 1H), 7.25 (dd, 1H), 6.07 (bt, 1H), 4.82 (m, 1H), 4.1 (t, 1H), 3.82 (dd, 1H), 3.6-3.75 (m, 2H), 2.65 (t, 3H), 2.04 (s, 3H).

### EXAMPLE 12

### N-(((5S)-3-(4((Z)-2bromo-2-(4-cyanophenyl)ethenyl)-3-fluorophenyl)-2-oxo-1,3-oxazolidin-5-yl)methyl)acetamide

The title compound was prepared according to the method described in Example 1 F, substituting (4-cyanophenyl)boronic acid for 5-acetylthiophene-2-boronic acid to afford the desired product. MS (ESI): m/e 459 (M+1)⁺; ¹H NMR (300 MHz, CDCl₃) δ8.06 (t, 1H), 7.77 (d, 2H), 7.68 (d, 2H), 7.57 (dd, 1H), 7.36 (s, 1H), 7.25 (dd, 1H), 5.96 (bt, 1H), 4.8 (m, 1H), 4.09 (t, 1H), 3.82 (dd, 1H), 3.6-3.75 (m, 2H), 2.04 (s, 3H).

### EXAMPLE 13

### N-(((5S)-3-(4-((Z)-2-bromo-2(4-isoquinolinyl)ethenyl)-3-fluorophenyl)-2-oxo-1,3-oxazolidin-5-yl)methyl)acetamide

The title compound was prepared according to the method described in Example 1F, substituting quinloline-3-boronic acid for 5-acetylthiophene-2-boronic acid to afford the desired product. MS (ESI): m/e 485 (M+1)⁺; ¹H NMR (300 MHz, CDCl₃) δ9.34 (d, 1H), 8.67 (s, 1H), 8.39 (bd, 1H), 8.09 (t, 1H), 8.01 (d, 1H), 7.89 (dt, 1H), 7.72 (dt, 1H), 7.57 (dd, 1H), 7.53 (s, 1H), 7.27 (dd, 1H), 5.97 (t, 1H), 4.81 (m, 1H), 4.11 (t, 1H), 3.83 (dd, 1H), 3.6-3.8 (m, 2H), 2.05 (s, 3H).

### EXAMPLE 14

### N-(((5S)-3-(4-((Z)-2-bromo-2-(4-pyridinyl)ethenyl)-3fluorophenyl)-2-oxo-1,3-oxazolidin-5-yl)methyl)acetamide

The title compound was prepared according to the method described in Example 1F, substituting pyridine-4-boronic acid for 5-acetylthiophene-2-boronic acid to afford the desired product. MS (ESI): m/e 435 (M+1)⁺; ¹H NMR (300 MHz, CDCl₃) δ10.25 (s, 1H), 8.7-8.8 (m, 1H), 8.2 (bt, 1H), 8.0 (bd, 1H), 7.8 (t, 1H), 7.4-7.7 (m, 2H), 7.2 (dd, 1H), 5.97 (t, 1H), 4.81 (m, 1H), 4.11 (t, 1H), 3.83 (dd, 1H), 3.6-3.8 (m, 2H), 2.05 (s, 3H).

### EXAMPLE 15

### methyl 3-((E)-2-(4-((5S)-5-((acetylamino)methyl)-2-oxo-1,3-oxazolidin-3yl)-2-fluorophenyl)ethenyl)benzoate

The title compound was prepared according to the method described in Example 1F, substituting (3-methoxycarbonylphenyl)boronic acid for 5-acetylthiophene-2-boronic acid to afford the product as a minor product. MS (ESI): m/e 413 (M+1)⁺; ¹H NMR (300 MHz, CDCl₃) δ8.2 (s, 1H), 7.92 (dm, 1H), 7.7 (dm, 1H), 7.4-7.6 (m, 4H), 7.1-7.3 (m, 2H), 6.0 (bt, 1H), 4.8 (m, 1H), 4.08 (t, 1H), 3.95 (s, 3H), 3.6-3.8 (m, 3H), 2.05 (s, 3H).

### EXAMPLE 16

### (2E)-3-(4-((Z)-2-(4-((5S)-5-((acetylamino)methyl)-2-oxo-1,3-oxazolidin-3-yl)-2-fluorophenyl)-1-bromoethenyl)phenyl)-2-propenoic acid

The title compound was prepared according to the method described in Example 1F, substituting 4-(2-carboxy-vinyl)phenyl-3-boronic acid for 5-acetylthiophene-2-boronic acid to afford the desired product. MS (ESI): m/e 504 (M+1)⁺; ¹H NMR (300 MHz, CDCl₃) δ8.25 (t, 1H), 7.87 (t, 1H), 7.78 (s, 3H), 7.5-7.7 (m, 3H), 7.4 (dd, 1H), 6.6 (d, 1H), 4.75 (m, 1H), 4.15 (t, 1H), 3.8 (dd, 1H), 3.45 (m, 2H), 1.83 (s, 3H).

### EXAMPLE 18

### methyl 3-((Z)-2-(4-((5S)-5-((acetylamino)methyl)-2-oxo-1,3-oxazolidin-3-yl)-2-fluorophenyl)-1-bromoethenyl)benzoate

The title compound was prepared according to the method described in Example 6, substituting carboxymethylphenyl-3-boronic acid for 5-acetylthiophene-2-boronic acid to afford the desired product. MS (ESI): m/e 492 (M+1)⁺.

### EXAMPLE 19

### N-(((5S)-3-(4-((Z)-2-(4-acetylphenyl)-2-bromoethyl)-3-fluorophenyl)-2-oxo-1,3-oxazolidin-5-yl)methyl)acetamide

The title compound was prepared according to the method described in Example 1F, substituting 4-acetylphenylboronic acid for 5-acetylthiophene-2-boronic acid to afford the desired product. MS (ESI): m/e 476 (M+1)⁺; ¹H NMR (300 MHz, CDCl₃) δ8.06 (t, 1H), 7.96 (t, 2H), 7.75 (d, 1H), 7.62 (d, 1H), 7.5-7.6 (m, 1H), 7.36 (s, 1H), 7.25 (m, 1H), 5.96 (bt, 1H), 4.8 (m, 1H), 4.09 (dt, 1H), 3.82 (dd, 1H), 3.6-3.75 (m, 2H), 2.64 (dd, 3H), 2.04 (s, 3H).

### EXAMPLE 20

### N-(((5S)-3-(3-fluoro-4-((E)-2-fluoro-2-(phenylsulfonyl)vinyl)phenyl)-2-oxo-1,3-oxazolidin-5-yl)methyl)acetamide

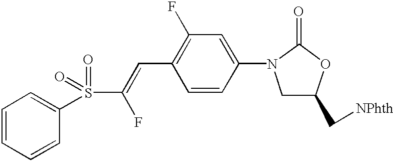

### EXAMPLE 20A

A −78° C. THF (70 mL) solution of fluoromethyl phenyl sulfone (6.15 g, prepared according to Org. Syn. 1993, 72, 216) and diethyl chlorophosphate (5.1 mL, 35 mmol) was treated with lithium bis(trimethylsilyl)acetamide (60 mL of a 1.0 M solution in THF, 60 mmol) added over a period of 20 minutes. The reaction was allowed to stir at −78° C. for an additional 60 minutes. Example 1C (10 g, 27 mmol) was added portion-wise as a solid over a period of 5 minutes. The reaction was allowed to slowly warm to room temperature with continued stirring for 16 hours. Next, the mixture was concentrated to remove 70% of the THF, then diluted with ethyl acetate (200 mL), and washed with saturated aqueous ammonium chloride solution (200 mL). The mixture was filtered to remove the desired product (8.4 g) that was >98% pure. The resultant liquor was separated and the aqueous layer was extracted with ethyl acetate (3×200 mL). The extract was dried over Na₂SO₄, filtered and concentrated. The dark yellow solid was triturated with diethyl ether (3×200 mL) to yield a second crop of the desired material (6.0 g) that was >95% pure.

MS (ESI(+)) m/e 525(M+H)⁺; ¹H NMR (300 MHz, DMSO-d₆) δ8.04 (m, 1H), 7.89 (m, 4H), 7.76 (m, 3H), 7.60 (dd, J=13.2, 2.4 Hz, 1H), 7.39 (dd, J=8.8, 2.0 Hz, 1H), 7.25 (d, J=35.6 Hz, 1H), 7.22 (2H), 4.98 (m, 1H), 4.24 (t, J=9.0 Hz, 1H), 3.95 (m, 3H).

### EXAMPLE 20B

### N-(((5S)-3-(3-fluoro-4-((E)-2-fluoro-2-(phenylsulfonyl)ethenyl)phenyl)-2-oxo-1,3-oxazolidin-5-yl)methyl)acetamide

Example 20A (8.4 g, 16 mmol) was dissolved in a solution of THF/ethanol (84 mL of a 5:3 mixture respectively) and treated with hydrazine hydrate (1.6 mL, 32 mmol). The reaction mixture was heated to 65° C. and was stirred for 2 hours. The mixture was cooled to room temperature and filtered. The solid material was washed with ethanol (2×25 mL) and the combined ethanol layers were concentrated. The resultant oil was dissolved in dichloromethane and pyridine (60 mL of a 4:1 mixture respectively) and treated with acetic anhydride (3.8 mL, 40 mmol). After I hour the reaction mixture was diluted with ethyl acetate (200 mL), then washed with a 1:1 saturated ammonium chloride/water mixture (100 mL) and a 1:1 saturated sodium chloride/water mixture (100 mL). The organic layer was concentrated, and the resultant material purified by flash column chromatography on silica gel with 1:1 hexanes/acetone as the eluent to provide the desired product.

MS (ESI(+)) m/e 437(M+H)⁺; ¹H NMR (300 MHz, DMSO-d₆) δ8.22 (t, J=5.8 Hz, 1H), 8.05 (m, 1H), 7.87 (m, 1H), 7.76 (m, 3H), 7.64 (dd, J=13.4, 2.2 Hz, 1H), 7.43 (dd, J=8.8, 2.0 Hz, 1H), 7.25 (d, J=35.9 Hz, 1H), 4.76 (m, 1H), 4.15 (t, J=9.2 Hz, 1H), 3.76 (dd, J=9.5, 6.4 Hz, 1H), 3.42 (t, J=5.6Hz, 2H), 1.82 (s, 3H).

### EXAMPLE 21

### N-(((5S)-3-(4-((Z)-2-bromo-2-(4-methoxy-3-pyridinyl)ethenyl)-3-fluorophenyl)-2-oxo-1,3-oxazolidin-5-yl)methyl)acetamide

The title compound was prepared according to the method described in Example 1F, substituting 2-methoxypyridyl-5-boronic acid for 5-acetylthiophene-2-boronic acid to afford the desired product. MS (ESI): m/e 465 (M+1)⁺; ¹H NMR (300 MHz, CDCl₃) δ8.44 (d, 1H), 8.02 (t, 1H), 7.85 (dd, 1H), 7.52 (dd, 1H), 7.23 (dd, 1H), 7.17 (s, 1H), 6.75 (dd, 1H), 5.95 (bt, 1H), 4.8 (m, 1H), 4.08 (t, 1H), 3.98 (s, 3H), 3.81 (dd, 1H), 3.6-3.75 (m, 2H), 2.03 (s, 3H).

### EXAMPLE 22

### N-(3((Z)-2-(4-((5S)-5-((acetylamino)methyl)-2-oxo-1,3 oxazolidin-3-yl)-2-fluorophenyl)-1-bromoethenyl)phenyl)acetamide

The title compound was prepared according to the method described in Example 1F, substituting 3-acetamidophenylboronic acid for 5-acetylthiophene-2-boronic acid to afford the desired product. MS (ESI): m/e 491 (M+1)⁺; ¹H NMR (300 MHz, CD₃OD) δ7.97 (t, 1H), 7.92 (t, 1H), 7.66-7.65 (m, 2H), 7.3-7.45 (m, 4H),4.8 (m, 1H), 4.19 (t, 1H), 3.86 (dd, 1H), 3.57 (d, 2H), 2.14 (s, 3H), 1.97 (s, 3H).

### EXAMPLE 23

### N-(((5S)-3-(3-fluoro-4-((E)-2-pyridin-3-ylethenyl)phenyl)-2-oxo-1,3-oxazolidin-5-yl)methyl)acetamide

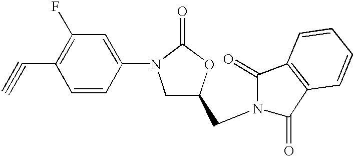

### EXAMPLE 23A

To a −78° C. solution of potassium t-botoxide (7.7 mL of a 1.0 M solution in THF) was added a −78° C. THF (4 mL) solution of dimethyl diazomethylphosphonate (1.15 g, prepared according to the procedure described in J. Org. Chem. 1971, 36, 1379-1385). The mixture was stirred at −78° C. for 10 minutes, then a −78° C. solution of Example 1C (2.18 g) in THF (100 mL) was added via cannula over 20 minutes. The reaction mixture was stirred for 24 hours gradually warming to room temperature. The mixture was quenched with 1:1 saturated ammonium chloride/ water (100 mL) and extracted with dichloromethane (50 mL). The aqueous layer was extracted with 4:1 THF/dichloromethane (3×100 mL). The extract was washed with 1:1 saturated sodium bicarbonate/water (100 mL), 1:1 brine/water (100 mL), dried (MgSO₄), filtered, and concentrated to provide crude product. The crude product was purified by flash column chromatography on silica gel with 98:2 dichloromethane/methanol to provide the desired product.

MS (ESI(+)) m/e 365 (M+H)⁺; ¹H NMR (300 MHz, DMSO-d₆) δ7.85-7.95 (m, 4H), 7.45-7.64 (m, 2H), 7.25-7.35 (dd, J=2.1, 8.7 Hz, 1H), 4.97 (m, 1H), 4.22 (t, J=9 Hz, 1H), 3.9-4.1 (m, 3H), 3.30 (s, 1H).

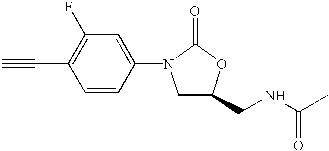

### EXAMPLE 23B

A suspension of Example 23A (1.65 g) in 1:1 THF/ethanol (34 mL) at 50° C. was treated with hydrazine monohydrate (3×240 μL) at 1 hour intervals. One hour after the final addition the mixture was cooled to 25° C. and filtered. The filtrate was concentrated in vacuo. The resultant material was dissolved in a mixture of pyridine (17 mL) and dichloromethane (17 mL), cooled to −5° C., and treated with acetic anhydride (854 μL). The reaction mixture was warmed to room temperature, stirred for 5 minutes and concentrated to provide crude product. The crude product was dissolved in 4:1 THF/dichloromethane (75 mL) and washed with 1:1 brine/water. The aqueous wash was extracted with dichloromethane (25 mL), dried (MgSO₄), diluted in acetone (20 mL) and filtered, and concentrated. The residue was triturated with hexanes to provide the desired product.

MS (ESI(+)) m/e 277 (M+H)⁺; ¹H NMR (300 MHz, CDCl₃) δ7.5-7.4 (m, 2H), 7.16-7.19 (dd, J=2.4, 8.4 Hz, 1H), 5.93 (bt, 1H), 4.8 (m, 1H), 4.05 (t, J=9 Hz, 1H), 3.8-3.6 (m, 3H), 3.28 (s, 1H), 2.02 (s, 3H).

### EXAMPLE 23C

### N-(((5S)-3-(3-fluoro-4-((E)-2-pyridin-3-ylvinyl)phenyl)-2-oxo-1,3-oxazolidin-5-yl)methyl)acetamide

A −15° C. solution of Example 23B (300 mg) in THF (1.5 mL) was treated with a solution of diisoamylborane in THF (2.65 mL, prepared according to the procedure described in J. Chem. Soc., Perkin Trans. 1, 1995, 23, 2955-2956) and stirred at −15° C. for 30 minutes. Next the mixture was warmed to 0° C. and stirred for 1 hour. The prepared solution was deoxygenated with nitrogen, treated sequentially with 3-bromopyridine (125 μL), tetrakis(triphenylphosphine)palladium (125 mg) and deoxygenated potassium carbonate solution (2.35 mL of a 0.725 M solution in water). The tube was sealed, heated to 70° C., and the reaction mixture was stirred for 20 hours. Then the mixture was cooled and poured into 1:1 saturated ammonium chloride/water (10 mL) and extracted with dichloromethane (3×10 mL). The extract was dried (MgSO₄), filtered, and concentrated. The concentrate was purified by flash column chromatography on silica gel with 95:5 dichloromethane/methanol to provide the desired product.

MS (ESI(+)) m/e 356 (M+H)⁺; ¹H NMR (300 MHz, DMSO-d₆) δ8.77 (d, J=1.8 Hz, 1H), 8.47 (dd, J=1.5, 4.8 Hz, 1H), 8.24 (t, J=5.7 Hz, 1H), 8.07 (dt, J=7.8, 2.1 Hz, 1H), 7.82 (t, J=8.7 Hz, 1H), 7.55 (dd, J=2.4, 13.5 Hz, 1H), 7.35-7.43 (m, 3H), 7.29 (d, J=16.8 Hz, 1H), 4.76 (m, 1H), 4.16 (t, J=9 Hz, 1H), 3.78 (dd, J=6.3, 9 Hz, 1H), 3.43 (t, J=5.4Hz, 2H), 1.89 (s, 3H).

### EXAMPLE 24

### N-(((5S)-3-(4-((E)-2-(5-cyanopyridin-3-yl)ethenyl)-3fluorophenyl)-2-oxo-1,3-oxazolidin-5-yl)methyl)acetamide

The desired product was prepared by substituting 5-bromonicotinonitrile for 3-bromopyridine in Example 23C.

MS (ESI(+)) m/e 381 (M+H)⁺; ¹H NMR (300 MHz, DMSO-d₆) δ9.04 (d, J=2.1 Hz, 1H), 8.88 (d, J=1.8 Hz, 1H), 8.24 (t, J=5.7 Hz, 1H), 7.8 (t, J=8.7 Hz, 1H), 7.56 (dd, J=2.4, 13.5 Hz, 1H), 7.55 (d, J=17.1 Hz, 1H), 7.4 (dd, J=1.8, 8.4 Hz, 1H), 7.33 (d, J=16.5 Hz, 1H), 4.76 (m, 1H), 4.16 (t, J=9 Hz, 1H), 3.78 (dd, J=6.6, 9 Hz, 1H), 3.43 (t, J=5.4 Hz, 2H), 1.89 (s, 3H).

### EXAMPLE 25

### N-(((5S)-3-(4-((1E)-2-(5-cyanopyridin-3-yl)prop-1-enyl)-3-fluorophenyl)-2-oxo-1,3-oxazolidin-5-yl)methyl)acetamide

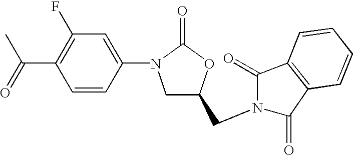

### EXAMPLE 25A

Example 1C (5 g) in dichloromethane (130 mL) was treated sequentially with diazomethane (3×60 mL of a 0.4 M solution in diethyl ether) at 24 hours intervals. The mixture was stirred at room temperature for 5 days, then concentrated and used directly in the next step.

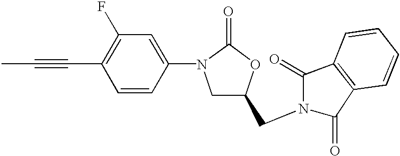

### EXAMPLE 25B

To −78° C. solution of potassium tert-butixide (17 mL of a 1.0 M solution in THF) was added a −78° C. THF (4 mL) solution of dimethyl diazomethylphosphonate (2.53 g, prepared according to the procedure described in J. Org. Chem. 1971, 36, 1379-1385). The mixture was stirred at −78° C. for 10 minutes; then a −78° C. solution of Example 25A (5.19 g) in THF (100 mL) was added via cannula over 20 minutes. The reaction mixture was stirred for 24 hours gradually warming to room temperature. The mixture was quenched with 1:1 saturated ammonium chloride/ water (200 mL) and extracted with dichloromethane (50 mL). The aqueous layer was extracted with 4:1 THF/dichloromethane (250 mL), and the combined extracts were washed with 1:1 saturated sodium bicarbonate/water (200 mL) and 1:1 brine/water (200 mL). The organic extracts were dried over MgSO₄, filtered, and concentrated to provide crude product. The crude product was purified by flash column chromatography on silica gel with 4:1 hexanes/acetone to provide the desired product.

MS (ESI(+)) m/e 379 (M+H)⁺; ¹H NMR (300 MHz, CDCl₃) δ7.9 (m, 2H), 7.77 (m, 2H), 7.45 (dd, J=2.4, 9 Hz, 1H), 7.36 (d, J=7.2 Hz, 1H), 7.25-7.35 (dd, J=2.4, 8.7 Hz, 1H), 4.97 (m, 1H), 4.07-4.18 (m, 2H), 4.01-3.87 (m, 2H), 2.09 (s, 3H).

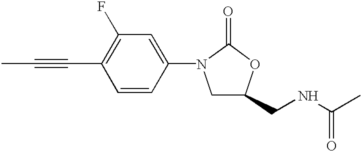

### EXAMPLE 25C

A 50° C. suspension of Example 25B (2.0 g) in 1:1 THF/ethanol (20 mL) was treated with hydrazine monohydrate (3×240 μL) at 1 hour intervals. One hour after the final addition the mixture was cooled to 25° C. and filtered. The filtrate was concentrated in vacuo. The resultant material was dissolved in a mixture of pyridine (10 mL) and dichloromethane (10 mL), cooled to −5° C., and treated with acetic anhydride (990 μL). The reaction mixture was warmed to room temperature, stirred for 5 minutes, and concentrated to provide crude product. The crude product was dissolved in 4:1 THF/dichloromethane (75 mL) and washed with 1:1 brine/water. The aqueous wash was extracted with dichloromethane (25 mL), and the combined extracts were dried over MgSO₄. Next the mixture was filtered and concentrated. The residue was dissolved in acetone (20 mL) and filtered. The filtrate was concentrated and the residue triturated with hexanes to provide the desired product.

MS (ESI(+)) m/e 291 (M+H)⁺; ¹H NMR (300 MHz, DMSO-d₆) δ8.22 (t, J=5.7 Hz, 1H), 7.56 (dd, J=2.1, 12.3 Hz, 1H), 7.47 (t, J=8.7 Hz, 1H), 7.3 (dd, J=2.1, 8.7 Hz, 1H), 4.73 (m, 1H), 4.12 (t, J=9 Hz, 1H), 3.74 (dd, J=6.3, 9.6 Hz, 1H), 3.41 (t, J=5.7 Hz, 2H), 2.07 (s, 3H), 1.83 (s, 3H)

### EXAMPLE 25D

### N-(((5S)-3-(4-((1E)-1-(5-cyanopyridin-3-yl)prop-1-enyl)-3-fluorophenyl)-2-oxo-1,3-oxazolidin-5-yl)methyl)acetamide

A −15° C. solution of Example 3C (350 mg) in THF (2.4 mL) was treated with a solution of diisoamylborane in THF (2.93 mL, prepared according to the procedure described in J. Chem. Soc., Perkin Trans. 1, 1995, 23, 2955-2956) and stirred at −15° C. for 30 minutes. Next the mixture was warmed to 0° C. and stirred for 1 hour. The prepared solution was deoxygenated with nitrogen, treated sequentially with 5-bromo-nicotinonitrile (264 mg), tetrakis(triphenylphosphine)palladium (140 mg) and deoxygenated potassium carbonate solution (2.60 mL of a 0.725 M solution in water). The tube was sealed, heated to 70° C., and the reaction mixture was stirred for 20 hours. Then the mixture was cooled and poured into 1:1 saturated ammonium chloride/water (10 mL) and extracted with dichloromethane (3×10 mL). The combined extracts were dried over MgSO₄, filtered, and concentrated to provide a mixture of isomers. The concentrate was purified by flash column chromatography on silica gel with 1:1 hexanes/acetone as the eluent to provide the desired product.

MS (ESI(+)) m/e 395 (M+H)⁺; ¹H NMR (300 MHz, DMSO-d₆) δ8.98 (d, J=2.4 Hz, 1H), 8.8 (d, J=1.8 Hz, 1H), 8.05 (t, J=2.1 Hz, 1H), 7.5 (dd, J=2.1, 12.3 Hz, 1H), 7.36 (t, J=8.4 Hz, 1H), 7.25 (dd, J=2.4 Hz, 1H), 6.84 (s, 1H), 5.95 (bt, 1H), 4.82 (m, 1H), 4.09 (t, J=9 Hz, 1H), 3.82 (dd, J=6.6, 9 Hz, 1H), 3.6-3.8 (m, 2H), 2.23 (s, 3H), 2.08 (s, 3H).

### EXAMPLE 26

### N-(((5S)-3-(4-((1Z)-1-(5-cyanopyridin-3-yl)prop-1-enyl)-3-fluorophenyl)-2-oxo-1,3-oxazolidin-5-yl)methyl)acetamide

The crude mixture of Example 25D was chromatographed as in Example 25D to give, in addition, the desired product as the title compound.

MS (ESI(+)) m/e 395 (M+H)⁺; ¹H NMR (300 MHz, DMSO-d₆) δ8.7 (t, J=2 Hz, 1H), 7.66 (t, J=2.1 Hz, 1H), 7.54 (dd, J=2.1, 12 Hz, 1H), 7.3 (dd, J=2.1, 8.4 Hz, 1H), 7.24 (s, 1H), 7.14 (t, J=8.1 Hz, 1H), 6.44 (q, J=6.9 Hz, 1H), 5.95 (bt, 1H), 4.82 (m, 1H), 4.1 (t, J=9 Hz, 1H), 3.81 (dd, J=6.6, 9 Hz, 1H), 3.6-3.8 (m, 2H), 2.04 (s, 3H), 1.78 (d, J=6.9 Hz, 3H).

### EXAMPLE 27

### (5S)-3-(4-((Z)-2-bromo-2-pyridin-3-ylethenyl)-3-fluorophenyl)-5-((isoxazol-3-ylamino)methyl)-1,3-oxazolidin-2-one

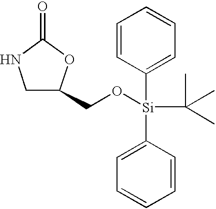

### EXAMPLE 27A

A 0° C. solution of (5R)-5-(hydroxymethyl)-1,3-oxazolidin-2-one (20.0 g, prepared according to the procedure described in Tetrahedron: Asymmetry 1995, 6, 1181-1190) in DMF (400 mL) was treated with imidazole (25.6 g), then tert-butylchlorodiphenylsilane (56.4 g) and allowed to stir at 20° C. for 20 hours. The reaction mixture was diluted with diethyl ether (200 mL) and washed with 1:1 brine/water (200 mL). The aqueous washes were extracted with diethyl ether (2×100 mL). The combined extracts were dried over MgSO₄, filtered and concentrated to provide crude product. The crude product was purified by flash column chromatography on silica gel with 4:1 hexanes/acetone as the eluent.

MS (ESI(+)) m/e 356 (M+H)⁺; ¹H NMR (300 MHz, CDCl₃) δ7.66 (m, 4H), 7.4 (m, 6H), 5.23 (bs, 1H), 4.68 (m, 1H), 3.86 (dd, J=4.5, 11.4 Hz, 1H), 3.75 (dd, J=3.9, 11.4 Hz, 1H), 3.57-3.67 (m, 2H), 1.06 (s, 9H).

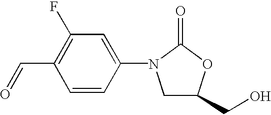

### EXAMPLE 27B

A suspension of Example 27A (3.55 g) in deoxygenated toluene (20 mL) was treated sequentially with 4-bromo-2-fluorobenzaldehyde (2.03 g), BINAP (250 mg), cesium carbonate (4.24 g), and tris(dibenzylideneacetone)dipalladium (183 mg). The tube was sealed, then heated to 100° C. and allowed to stir for 4 hours. Next, the mixture was cooled to room temperature, poured into 1:1 saturated ammonium chloride/water (200 mL) and extracted with ethyl acetate (3×100 mL). The combined extracts were dried over MgSO₄, filtered, and concentrated. The concentrate was purified by flash column chromatography on silica gel with 2:1 hexanes/acetone as the eluent to provide the silyl-protected product. The product was treated with tetrabutylammonium fluoride (4 mL of a 1.0 M solution in THF) and stirred at room temperature for 20 minutes. The reaction mixture was quenched with 1:1 saturated ammonium chloride/water (20 mL) and extracted with ethyl acetate (3×20 mL). The extract was dried over MgSO₄, filtered, and concentrated. The crude product was purified by flash column chromatography on silica gel with 3:2 hexanes/acetone as the eluent to provide the desired product.

MS (ESI(+)) m/e 240 (M+H)⁺; ¹H NMR (300 MHz, CDCl₃) δ10.25 (s, 1H), 7.89 (t, J=8.1 Hz, 1H), 7.67 (dd, J=12.9, 2.1 Hz, 1H), 7.3 (dd, J=8.7, 2.4 Hz, 1H), 4.8 (m, 1H), 4.05 (m, 3H), 3.8 (dd, J=12.6 3.3 Hz, 1H).

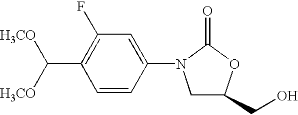

### EXAMPLE 27C

A solution of Example 27B (630 mg) in anhydrous methanol (13 mL) was treated with p-toluenesulfonic acid (2 mg) and trimethylorthoformate (560 μL), and the resultant mixture was stirred for 1 hour. The reaction mixture was washed with saturated sodium bicarbonate (10 mL) and extracted with dichloromethane (2× mL). The combined extracts were dried over MgSO₄, filtered and concentrated. The concentrate was purified by flash column chromatography on silica gel with 1:2 hexanes/acetone as the eluent to provide the desired product.

MS (ESI(+)) m/e 284 (MH)⁺; ¹H NMR (300 MHz, CDCl₃) δ7.53 (dd, J=2.1, 17.6 Hz, 1H), 7.5 (d, J=8.7, 1H), 7.35 (dd, J=2.1, 8.7 Hz, 1H), 5.54 (s, 1H), 5.2 (t, J=8.7 Hz, 1H), 4.7 (m, 1H), 4.09 (t, J=9 Hz, 1H), 3.52-3.71 (dm, 2H), 3.26 (s, 6H).

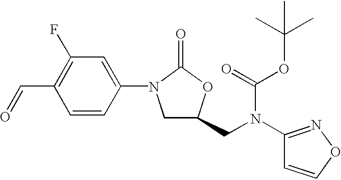

### EXAMPLE 27D

Polymer-supported triphenylphosphine (1.2 g of 3.0 mmol/g loading, 2% divinylbenzene cross-linked polystyrene resin) was swelled in THF (14 mL) and treated with Example 27C (830 mg) and isoxazol-3-yl-carbamic acid tert-butyl ester (804 mg, prepared according to the procedure described in Tetrahedron Letters 1996, 37, 3339-3342). Next the mixture was cooled to −5° C. and treated under nitrogen with a solution of di-tert-butyl azodicarboxylate (804 mg) in THF (4 mL). After stirring at room temperature for 24 hours, the reaction mixture was filtered and the resin was washed sequentially with dichloromethane (50 mL), 1:1 dichloromethane/methanol (50 mL), and methanol (50 mL). The combined extracts were concentrated and purified by flash column chromatography on silica gel with 3:1 hexanes/acetone as the eluent to provide the protected product. This material was dissolved in 98:2 acetonitrile/water (10 mL), treated with trifluoroacetic acid (100 μL), and stirred for 30 minutes. The reaction mixture was taken into toluene (10 mL), concentrated and used directly in the next step.

MS (ESI(+)) m/e 406 (M+H)⁺

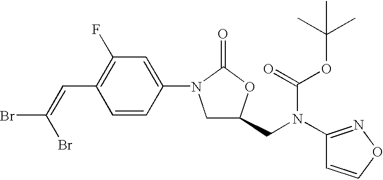

### EXAMPLE 27E

Polymer-supported triphenylphosphine (5.0 g of 3 mmol/g loading, 2% divinylbenzene cross-linked polystyrene resin) was swelled in dichloromethane (50 mL) and cooled to −10° C. The mixture was treated with carbon tetrabromide (2.5 g), warmed to −5° C., and stirred for 30 minutes. Next the mixture was treated portion wise with Example 27D (1 g) and stirred for an additional 20 minutes. The mixture was quenched with methanol (50 mL) and filtered. The resin was washed sequentially with dichloromethane (100 mL), 1:1 dichloromethane/methanol (100 mL), and methanol (3×100 mL). The filtrate and washes were combined and concentrated. The concentrate was dissolved in dichloromethane (150 mL) and washed with 1:1 saturated sodium bicarbonate/water and 1:1 brine/water. The aqueous washes were extracted with dichloromethane (2×50 mL), and the extract was dried over MgSO₄, filtered and concentrated. The concentrate was purified by flash column chromatography on silica gel with 1:1 hexanes/acetone as the eluent to provide the desired product.

MS (ESI(+)) m/e 562 (M+H)⁺; ¹H NMR (300 MHz, CDCl₃) δ8.26 (d, J=1.8 Hz, 1H), 7.82 (t, J=8.4 Hz, 1H), 7.5 (m, 2H), 7.25 (m, 2H), 5.09 (m, 1H), 4.38 (dd, J=15, 7.8 Hz, 1H), 4.12 (m, 2 H), 3.83 (dd, J=9.3, 5.4 Hz, 1H), 1.55 (s, 9H).

### EXAMPLE 27F

### (5S)-3-(4-((Z)-2-bromo-2-pyridin-3-ylvinyl)-3-fluorophenyl)-5-((isoxazol-3-ylamino)methyl)-1,3-oxazolidin-2-one

A solution of Example 27E (200 mg) in THF (1.5 mL) was sparged with nitrogen, then treated sequentially with 3-pyridylboronic acid, tetrakis(triphenylphosphine)palladium (41 mg) and deoxygenated potassium carbonate solution (0.52 mL of a 0.725 M solution in water). The reaction vessel was sealed, heated to 70° C., and the contents were stirred for 20 hours. The mixture was cooled to room temperature, poured into 1:1 saturated ammonium chloride/water (10 mL) and extracted with dichloromethane (3×10 mL). The combined extracts were dried over MgSO₄, filtered and concentrated. The concentrate was purified with flash column chromatography on silica gel with 95:5 dichloromethane/methanol as the eluent to provide the protected product. Next, the product was dissolved in a 4.0 N solution of hydrochloric acid in dioxane (2 mL) and stirred at room temperature for 1 hour. The mixture was concentrated to provide the desired product.

MS (ESI(+)) m/e 460 (M+H)⁺; ¹H NMR (300 MHz, DMSO-d₆) δ8.96 (d, J=1.8 Hz, 1H), 8.64 (dd, J=1.5, 4.5 Hz, 1H), 8.4 (d, J=2.1 Hz, 1H), 8.17 (td, J=1.5, 4.5 Hz, 1H), 7.88 (t, J=8.7 Hz, 1H), 7.4-7.7 (m, 5H), 6.01 (d, J=2.1 Hz, 1H), 4.76 (m, 1H), 4.21 (t, J=9 Hz, 1H), 3.78 (dd, J=6.3, 9.6 Hz, 1H), 3.42 (t, J=5.7 Hz, 2H).

EXAMPLES 28B and 28C

### N-(((5S)-3-(3-fluoro-4-((Z)-2-fluoro-2-pyridin-3-ylethenyl)phenyl)-2-oxo-1,3-oxazolidin-yl)methyl)acetamide

### and

### N-(((5S)-3-(3-fluoro-4-((E)-2-fluoro-2-pyridin-3-ylethenyl)phenyl)-2-oxo-1,3-oxazolidin-5-yl)methyl)acetamide

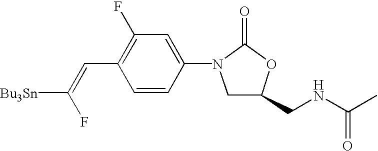

### EXAMPLE 28A

Example 20B (1.8 g, 4.1 mmol) as a suspension in benzene (80 mL) was treated with tributyltin hydride (3.3 mL, 12.4 mmol) and AIBN (60 mg, 0.4 mmol). The mixture was heated to 90° C. and was stirred for 2 hours. The reaction mixture was cooled to room temperature and concentrated in vacuo. The resultant semi-solid was purified by flash column chromatography on silica gel with 2:1 hexanes/acetone as the eluent to provide the desired product.

MS (ESI(+)) m/e 585(M)⁺; ¹H NMR (300 MHz, DMSO-d₆) δ8.23 (t, J=5.8 Hz, 1H), 7.80 (t, J=8.8 Hz, 1 H), 7.52 (dd J=13.2, 2.4Hz, 1H), 7.28 (dd, J=8.6, 1.9 Hz, 1H), 5.90 (d, J=58.0 Hz, 1H), 4.74 (m, 1H), 4.12 (t, J=9.2 Hz, 1H), 3.74 (dd, J=9.2, 6.4 Hz, 1H), 3.42 (t, J=5.6 Hz, 2H), 1.83 (s, 3H), 1.56 (m, 6H), 1.32 (m, 6H), 1.08 (t, J=7.8 Hz, 6H), 0.88 (t, J=7.3 Hz, 9H).

### EXAMPLES 28B and 28C

### (5S)-3-(4-((Z)-2-bromo-2-pyridin-3-ylvinyl)-3-fluorophenyl)-5-((isoxazol-3-ylamino)methyl)-1,3-oxazolidin-2-one

### and

### (5S)-3-(4-((E)-2-bromo-2-pyridin-3-ylvinyl)-3-fluorophenyl)-5-((isoxazol-3-ylamino)methyl)-1,3-oxazolidin-2-one

Example 28A (130 mg, 0.22 mmol), copper(I) iodide (40 mg, 0.21 mmol), and 3-bromopyridine (34 mg, 0.22 mmol) were placed in a flask under a nitrogen atmosphere. To this was added a mixture of tris(dibenzylideneacetone)dipalladium(0) (10.1 mg, 0.04 mmol) and tri-tert-butylphosphine (10 mg, 0.04 mmol) in deoxygenated dioxane (1.1 mL). The reaction mixture was sealed, heated to 75° C. and was stirred for 8 hours. The mixture was concentrated and purified by flash column chromatography on silica gel with 1:1 hexanes/acetone as the eluent to provide the desired products.

Data for Z isomer: MS (ESI(+)) m/e 374(M+H)⁺; ¹H NMR (300 MHz, DMSO-d₆) δ8.99 (s, 1H), 8.64 (d, J=4.4 Hz, 1H), 8.24 (t, J=5.7 Hz, 1H), 8.19 (d, J=8.1 Hz, 1H), 7.89 (t, J=8.8 Hz, 1H), 7.61 (dd, J=2.4, 13.6 Hz, 1H), 7.52 (dd, J=4.7, 8.1 Hz, 1H), 7.41 (dd, 2.4, 8.8 Hz, 1H), 6.87 (d, J=41 Hz, 1H), 4.76 (m, 1H), 4.16 (t, J=9.2 Hz, H), 3.79 (dd, J=6.5, 9.5 Hz, 1H), 3.43 (t, J=5.4 Hz, 2H), 1.84 (s, 3H).

Data for E isomer: MS (ESI(+)) m/e 374 (M+H)⁺; ¹H NMR (300 MHz, DMSO-d₆) δ8.60 (d, J=4.1 Hz, 1H), 8.51 (s, 1H), 8.22 (m, 2 H), 7.80 (dt, J=8.1, 1.7 Hz, 1H), 7.52 (dd, J=12.7, 2.2 Hz, 1H), 7.45 (dd, J=8.0, 4.9 Hz, 1H), 7.22 (d, J=2.0 Hz, 1H), 6.65 (d, J=20.3 Hz, 1H), 4.73 (m, 1H), 4.10 (t, J=9.2 Hz, 1H), 3.72 (dd, J=9.3, 6.6 Hz, 1H), 3.40 (t, J=5.9 Hz, 2H), 1.83 (s, 3H).

### EXAMPLE 30

### N-(((5S)-3-(3-fluoro-4-((Z)-2-fluoro-2-pyridin-5-ylphenyl)-2-oxo-1,3-oxazolidin-5-yl)methyl)acetamide

The desired product was prepared as in Example 28B substituting 5-bromopyrimidine for 3-bromopyridine.

MS (ESI(+)) m/e 375 (M+H)⁺; ¹H NMR (300 MHz, DMSO-d₆) δ9.21 (d, J=9.0 Hz, 2H), 8.24 (t, J=6.0 Hz, 1H), 7.90 (t, J=8.7 Hz, 1H), 7.61 (dd, J=13.3, 2.3 Hz, 1H), 7.42 (dd, J=8.9, 2.2 Hz, 1H), 7.00 (d, J=41.0 Hz, 1H), 4.78 (dd, J=16.5, 8.8 Hz, 1H), 4.76 (m, 1H), 4.16 (t, J=9.0 Hz, 1H), 3.78 (dd, J=9.2, 6.5 Hz, 1H), 3.43 (t, J=5.5 Hz, 2H), 1.84 (s, 3H).

### EXAMPLE 31

### N-(((5S)-3(3-fluoro-4-((Z)-2-fluoro-2-(1,3-thiazol-2-yl)ethenyl)phenyl)-2-oxo-1,3-oxazolidin-5-yl)methyl)acetamide

The desired product was prepared as in Example 28B substituting 2-bromothiazole for 3-bromopyridine.

MS (ESI(+)) m/e 380 (M+H)⁺; ¹H NMR (300 MHz, DMSO-d₆) δ8.24 (t, J=5.8 Hz, 1H), 8.02 (t, J=3.5 Hz, 1H), 7.97 (m, 1H), 7.91 (t, J=8.8 Hz, 1H), 7.62 (dd, J=2.4, 13.2 Hz, 1H), 7.43 (dd, J=2.0, 8.8 Hz, 1H), 7.00 (d, J=40 Hz, 1H), 4.77 (m, 1H), 4.16 (t, J=9.2 Hz, 1H), 3.79 (dd, J=6.5, 9.2 Hz, 1H), 3.43 (t, J=5.4 Hz, 2H), 1.84 (s, 3H).

### EXAMPLE 32

### N-(((5S)-3-(4-((Z)-2-(2-aminopyrimidin-5-yl)-2-fluoroethenyl)-3-fluorophenyl)-2-oxo-1,3-oxazolidin-5-yl)methyl)acetamide

The desired product was prepared as in Example 28B substituting 5-bromo-pyrimidin-2-ylamine for 3-bromopyridine.

MS (ESI(+)) m/e 390 (M+H)⁺; ¹H NMR (300 MHz, DMSO-d₆) δ8.21 (t, J=5.8 Hz, 1H), 8.16 (s, 1H), 7.52 (d, J=13.6 Hz, 1H), 7.24 (s, 2H), 7.17 (s, 1H), 6.37 (d, J=19 Hz, 1H), 4.72 (m, 1H), 4.11 (t, J=9.2 Hz, 1H), 3.73 (dd, J=6.5, 9.5 Hz, 1H), 3.43 (t, J=5.4 Hz, 2H), 1.83 (s, 3H).

### EXAMPLE 33

### N-(((5S)-3-(3-fluoro-4-((Z)-2-fluoro-2-(2-methyl-2H-tetraazol-5-yl)ethenyl)phenyl)2-oxo-1,3-oxazolidin-5-yl)methyl)acetamide

The desired product was prepared as in Example 28B substituting 5-iodo-2-methyl-2H-tetrazole for 3-bromopyridine.

MS (ESI(+)) m/e 379 (M+H)⁺; ¹H NMR (300 MHz, DMSO-d₆) δ8.24 (t, J=5.8 Hz, 1H), 7.94 (t, J=8.7 Hz, 1H), 7.63 (dd, J=2.4, 13.2 Hz, 1H), 7.45 (dd, J=2.0, 8.8 Hz, 1H), 7.00 (d, J=39 Hz, 1H), 4.76 (m, 1H), 4.44 (s, 3H), 4.17 (t, J=9.2 Hz, 1H), 3.79 (dd, J=6.5, 9.5 Hz, 1H), 3.43 (t, J=5.4 Hz, 2H), 1.84 (s, 3H).

### EXAMPLE 34

### N-(((5S)-3-(4-((Z)-2(2-cyanothien-3-yl)-2-fluoroethenyl)-3-fluorophenyl)-2-oxo1,3-oxazolidin-5-yl)methyl)acetamide

The desired product was prepared as in Example 28B substituting 3-bromo-thiophene-2-carbonitrile for 3-bromopyridine.

MS (ESI(+)) m/e 404 (M+H)⁺; ¹H NMR (300 MHz, DMSO-d₆) δ8.24 (t, J=5.8 Hz, 1H), 8.12 (dd, J=2, 5.4 Hz, 1H), 7.89 (t, J=8.8 Hz, 1H), 7.77 (dd, J=1.5, 4 Hz, 1H), 7.61 (dd, J=2.4, 13.2 Hz, 1H), 7.41 (dd, J=2.4, 8.8 Hz, 1H), 6.91 (d, J=40 Hz, 1H), 4.76 (m, 1H), 4.16 (t, J=9.2 Hz, 1H), 3.79 (dd, J=6.5, 9.2 Hz, 1H), 3.43 (t, J=5.7 Hz, 2H), 1.84 (s, 3H).

### EXAMPLE 35

### N-(((5S)-3-(4-((Z)-2-(5-cyanopyridin-3-yl)-2-fluoroethenyl)-3-fluorophenyl)-2-oxo-1,3-oxazolidin-5-yl)methyl)acetamide

The desired product was prepared as in Example 28B substituting 5-bromo-nicotinonitrile for 3-bromopyridine.

MS (ESI(+)) m/e 399 (M+H)⁺; ¹H NMR (300 MHz, DMSO-d₆) δ9.23 (d, J=2.4 Hz, 1H), 9.06 (d, J=2.0 Hz, 1H), 8.76 (t, J=2.0 Hz, 1 H), 8.24 (t, J=5.9 Hz, 1 H), 7.90 (t, J=8.8 Hz, 1 H), 7.62 (dd, J=13.3, 2.4 Hz, 1H), 7.42 (dd, J=8.8, 2.0 Hz, 1H), 7.07 (d, J=40.7 Hz, 1H), 4.77 (m, 1H), 4.17 (t, J=9.0 Hz, 1H), 3.78 (dd, J=9.3, 6.6 Hz, 1H), 3.44 (t, J=5.4 Hz, 2H), 1.84 (s, 3H).

### EXAMPLE 36

### N-(((5S)-3-(4-((Z)-2-(3-acetylphenyl)-2-fluoroethenyl)-3-fluorophenyl)-2-oxo-1,3-oxazolidin-5-yl)methyl)acetamide

The desired product was prepared as in Example 28B substituting 3-bromoacetophenone for 3-bromopyridine.

MS (ESI(+)) m/e 415 (M+H)⁺; ¹H NMR (300 MHz, DMSO-d₆) δ8.24 (m, 2H), 8.02 (dt, J=7.9, 1.7 Hz, 2H), 7.90 (t, J=8.8 Hz, 1 H), 7.66 (t, J=7.8 Hz, 1 H), 7.61 (dd, J=13.2, 2.3 Hz, 1 H), 7.40 (dd, J=8.8, 2.2 Hz, 1H), 6.86 (d, J=40.6 Hz, 1H), 4.76 (m, 1H), 4.16 (t, J=9.0 Hz, 1H), 3.78 (dd, J=9.2, 6.5Hz, 1H), 3.43 (t, J=5.5 Hz, 2H), 2.66 (s, 3H), 1.84 (s, 3H).

### EXAMPLE 37

### N-(((5S)-3-(3-fluoro-4-((Z)2-fluoro-2-(3formylphenyl)ethenyl)phenyl)-2-oxo-1,3-oxazolidin-5-yl)methyl)acetamide

The desired product was prepared as in Example 28B substituting 3-bromobenzaldehyde for 3-bromopyridine.

MS (ESI(+)) m/e 401 (M+H)⁺; ¹H NMR (300 MHz, DMSO-d₆) δ10.10 (s, 1 H), 8.28 (s, 1 H), 8.25 (t, J=5.9 Hz, 1H), 8.10 (d, J=7.8 Hz, 1H), 7.97 (d, J=7.8 Hz, 1H), 7.91 (t, J=8.8 Hz, 1H), 7.72 (t, J=7.6 Hz, 1H), 7.61 (dd, J=13.4, 2.2 Hz, 1H), 7.41 (dd, J=8.8, 2.0 Hz, 1H), 6.88 (d, J=40.7 Hz, 1H), 4.77 (m, 1H), 4.17 (t, J=9.0 Hz, 1H), 3.78 (dd, J=9.3, 6.6 Hz, 1H), 3.44 (t, J=5.4 Hz, 2H), 1.84 (s, 3 H).

### EXAMPLE 38

### N-(((5S)-3-(4-((Z)-2-(3-cyanophenyl)-2-fluorophenyl)-3-fluorophenyl)-2-oxo-1,3-oxazolidin-5-yl)methyl)acetamide

The desired product was prepared as in Example 28B substituting 3-bromo-benzonitrile for 3-bromopyridine. A crude product mixture was obtained which was chromatographed as above to afford the desired product.

MS (ESI(+)) m/e 398 (M+H)⁺; ¹H NMR (300 MHz, DMSO-d₆) δ8.29 (s, 1 H), 8.25 (t, J=5.8 Hz, 1 H), 8.08 (d, J=8.3 Hz, 1H), 7.89 (t, J=8.7 Hz, 1H), 7.89 (t, J=8.7 Hz, 2H), 7.61 (dd, J=13.2, 2.2 Hz, 1H), 7.41 (dd, J=8.7, 2.1 Hz, 1H), 6.94 (d, J=40.5 Hz, 1H), 4.76 (m, 1H), 4.16 (t, J=9.1 Hz, 1H), 3.78 (dd, J=9.2, 6.5 Hz, 1H), 3.43 (t, J=5.6 Hz, 2H), 1.84 (s, 3H).

### EXAMPLE 39

### N-(((5S)-3-(3-fluoro-4-((E/Z)-2-fluoroethenyl)phenyl)-2-oxo-1,3-oxazolidin-5-yl)methyl)acetamide

The crude mixture of Example 38 was chromatographed as in Example 38 to give, in addition, the desired product as the title compound.

MS (ESI(+)) m/e 297 (M+H)⁺; ¹H NMR (300 MHz, DMSO-d₆) δ8.23 (t, J=5.6 Hz, 1H), 7.73 (t, J=8.6 Hz, 1H), 7.55 (dd, J=13.2, 2.4 Hz, 1H), 7.34 (dd, J=8.6, 2.2 Hz, 1H), 7.02 (dd, J=83.2, 5.3 Hz, 1H), 5.91 (dd, J=45.9, 5.3 Hz, 1H), 4.75 (m, 1H), 4.13 (t, J=9.2 Hz, 1H), 3.75 (dd, J=9.2, 6.4 Hz, 1H), 3.42 (t, J=5.4 Hz, 2H), 1.83 (s, 3H).

### EXAMPLE 40

### N-(((5S)-3-(4((Z)-2-(3-aminophenyl)-2-bromophenyl)-3-fluorophenyl)-2-oxo-1,3-oxazolidin-5-yl)methyl)acetamide

A solution of Example 1E (41 mg) in deoxygenated DME (1.3 mL) was treated with 3-aminophenylboronic acid (17.5 mg), tetrakis(triphenylphosphine)palladium (6 mg) and a deoxygenated solution of potassium carbonate (0.1 mL of a 2 N solution in water). The reaction vessel was sealed and heated to 70° C. with stirring for 20 hours. Next, the mixture was cooled to room temperature and poured into 1:1 saturated ammonium chloride/water (5 mL) and extracted with dichloromethane (3×5 mL). The combined extracts were dried (MgSO₄), filtered, and concentrated. The concentrate was purified with flash column chromatography on silica gel with 1:1 hexane/acetone as eluent to provide the desired product.

MS (ESI(+)) m/e 448 (M+H)⁺; ¹H NMR (300 MHz, MeOH-d₄) δ7.98 (t, J=8.4 Hz, 1H), 7.68 (bd, 1H), 7.6 (m, 2H), 7.52 (t, J=8.1 Hz, 1H), 7.4 (s, 1H), 7.35 (dd, J=2.4, 9 Hz, 1H), 7.3 (bd, 1H), 4.8 (m, 1H), 4.19 (t, 9Hz, 1H), 3.86 (dd, J=6.6, 9 Hz, 1H), 3.58 (d, J=4.8 Hz, 2H), 1.97 (s, 3H).

### EXAMPLE 41

### N-(((5S)-3-(4-((Z)-2-bromo-2-(5-cyanopyridin-3-yl)ethenyl)-3-fluorophenyl)-2-oxo-1,3-oxazolidin-5-yl)methyl)acetamide

The desired product was prepared by substituting 5-(4,4,5,5-tetramethyl-(1,3,2)dioxaborolan-2-yl)-nicotinonitrile (27.6 mg, prepared according to the procedure described in Tetrahedron Letters, 1997, 38, 3447-3450) for 3-aminophenylboronic acid in Example 40.

MA (ESI(+)) m/e 459 (M+H)⁺; ¹H NMR (300 MHz, CDCl₃) δ9.08 (s, 1H), 8.83 (s, 1H), 8.23 (t, J=2.1 Hz, 1H), 8.07 (t, J=8.7 Hz, 1H), 7.58 (dd, J=2.1, 12.5 Hz, 1H), 7.39 (s, 1H), 7.28 (dd, J=2.1, 8.4 Hz, 1H), 4.8 (m, 1H), 4.08 (t, J=9 Hz, 1H), 3.83 (dd, J=6.6, 9.6 Hz, 1H), 3.6-3.75 (m, 2H), 2.04 (s, 3H).

### EXAMPLE 42

### methyl 3-((Z)-2-(4((5S)-5-((acetylamino)methyl)-2-oxo-1,3-oxazolidin-3-yl)-2-fluorophenyl)-1-bromoethenyl)-5-aminobenzoate

The desired product was prepared by substituting 3-amino-5-methoxycarbonylphenylboronic acid for 3-aminophenylboronic acid in Example 40.

MS (ESI(+)) m/e 506 (M+H)⁺; ¹H NMR (300 MHz, MeOH-d₄) δ7.98 (t, J=8.7 Hz, 1H), 7.86 (s, 1H), 7.6 (m, 2H), 7.47 (s, 1H), 7.33 (m, 2H), 4.8 (m, 1H), 4.18 (t, J=9 Hz, 1H), 3.92 (s, 3H), 3.86 (dd, J=6.6, 9 Hz, 1H), 3.58 (d, J=5.1 Hz, 2H), 1.97 (s, 3H).

### EXAMPLE 43

### N-(((5S)-3-(4-((Z)-2-(3-(aminomethyl)phenyl)-2-bromoethenyl)-3-fluorophenyl)-2-oxo-1,3-oxazolidin-5-yl)methyl)acetamide

The desired product was prepared by substituting 3-aminomethylphenylboronic acid for 3-aminophenylboronic acid in Example 40.

MS (ESI(+)) m/e 462 (M+H)⁺; ¹H NMR (300 MHz, MeOH-d₄) δ7.99 (t, J=8.7 Hz, 1H), 7.81 (s, 1H), 7.77 (dt, 1H), 7.6 (dd, J=2.4, 12.6 Hz, 1H), 7.5 (t, J=7.8 Hz, 1H), 7.47 (s, 1H), 7.38 (s, 1H), 7.35 (dd, J=2.1, 8.4 Hz, 1H), 4.8 (m, 1H), 4.16-4.12 (m, 1H), 4.19 (s, 2H), 3.87 (dd, J=6, 9 Hz, 1H), 3.58 (m, 2H), 1.96 (s, 3H).

### EXAMPLE 44

### N-(((5S)-3-(4-((Z)-2-bromo-2-phenylethenyl)-3-fluorophenyl)-2-oxo-1,3-oxazolidin-5-yl)methyl)acetamide

The desired product was prepared by substituting phenylboronic acid for 3-aminophenylboronic acid in Example 40.

MS (ESI(+)) m/e 434 (M+H)³⁰ ; ¹H NMR (300 MHz, DMSO-d₆) δ8.24 (t, J=5.8 Hz, 1H), 7.86 (t, J=8.7 Hz, 1H), 77.73 (d, J=1.7 Hz, 1H), 7.71 (d, J=1.4 Hz, 1H), 7.58 (dd, J=2.0, 12.9 Hz, 1H), 7.35-7.5 (m, 5H), 4.76 (m, 1H), 4.17 (t, J=9.2 Hz, 1H), 3.79 (dd, J=6.8, 9.2 Hz, 1H), 3.43 (t, J=5.7 Hz, 2H), 1.84 (s, 3H).

### EXAMPLE 45

### 3-((Z)-2-(4-((5S)-5-((acetylamino)methyl)-2-oxo-1,3-oxazolidin-3-yl)-2-fluorophenyl)-2-bromoethenyl)benzoic acid

A 25° C. solution of Example 8 (130 mg) in tert-butanol (6 mL) was treated with 2-methyl-2-butene (3 mL), followed by a solution of sodium chlorite (570 mg) and sodium dihydrogenphosphite (600 mg) in water (12 mL). The reaction mixture was stirred for 2 hours, then concentrated and diluted with ethyl acetate (10 mL). This mixture was washed with 1:1 saturated ammonium chloride/water (5 mL) and the aqueous layer was extracted with ethyl acetate (3×5 mL). The extract was dried over MgSO₄, filtered, and concentrated.

MS (ESI(+)) m/e 477 (M+H)⁺; ¹H NMR (300 MHz, MeOH-d₄) δ13.19 (s, 1H), 8.24 (m, 1 H), 7.97 (dd, J=1.8, 7.8 Hz, 2H), 7.87 (t, J=9 Hz, 1H), 7.61 (m, 3H), 7.52 (s, 1H), 7.41 (dd, J=2.1, 9.0 Hz, 1H), 4.77 (m, 1H), 4.17 (t, J=8.7 Hz, 1H), 3.78 (dd, J=6.6,9.3 Hz, 1H), 3.44 (t, J=5.4Hz, 2H), 1.84 (s, 3H).

### EXAMPLE 46

### 3-((Z)-2-(4-((5S)-5-((acetylamino)methyl)-2-oxo-1,3-oxazolidin-3yl)-2-fluorophenyl)-1-bromoethenyl)benzamide

A 25° C. solution of Example 45 (24.4 mg) in DMF (0.2 mL) was treated sequentially with EDAC (15 mg), 1-hydroxybenzotriazole hydrate (10 mg), N,N-diisopropylethylamine (0.035 mL) and ammonium chloride (6 mg). The mixture was stirred for 20 hours, then concentrated and diluted with dichloromethane (1 mL). This mixture was washed with 1:1 brine/water (5 mL) and the aqueous layer was extracted with dichloromethane (3×2 mL). The extract was dried over MgSO₄, filtered, and concentrated. The concentrate was purified with flash column chromatography on silica gel with 95:5 dichloromethane/methanol as the eluent to provide the desired product.

MS (ESI(+)) m/e 476 (M+H)⁺; ¹H NMR (300 MHz, MeOH-d₄) δ8.19 (t, J=1.8 Hz, 1H), 7.98 (t, J=8.4 Hz, 1H), 7.88 (m, 2H), 7.6 (dd, J=2.1, 12.9 Hz, 1H), 7.52 (t, J=8.1 Hz, 1H), 7.4 (s, 1H), 7.34 (dd, J=2.1, 8.7 Hz, 1H), 4.8 (m, 1H), 4.19 (t, J=9 Hz, 1H), 3.87 (dd, J=6, 9.3 Hz, 1H), 3.58 (d, J=4.8 Hz, 2H), 1.97 (s, 3H).

### EXAMPLE 47

### N-(((5S)-3-(4-((Z)-2-bromo-2-(3-((dimethylamino)methyl)phenyl)ethenyl)-3-fluorophenyl)-2-oxo-1,3-oxazolidin-5-yl)methyl)acetamide

A 25° C. solution of Example 8 (130 mg) in THF (0.29 mL) was treated sequentially with dimethylamine hydrochloride (9 mg), acetic acid (0.005 mL) and sodium triacetoxycyanoborohydride (28 mg). The reaction mixture was stirred for 4 hours, then concentrated and diluted with ethyl acetate (5 mL). This was then washed with 1:1 saturated solution of sodium bicarbonate/water (2 mL), and the aqueous layer was extracted with ethyl acetate (3×2 mL). The extract was dried over MgSO₄, filtered and concentrated. The concentrate was purified by flash column chromatography on silica gel with 9:1 dichloromethane/methanol as eluent to provide the desired product.

MS (ESI(+)) m/e 490 (M+H)⁺; ¹H NMR (300 MHz, MeOH-d₄) δ7.97 (t, J=8.4 Hz, IH), 7.55-7.67 (m, 3H), 7.33-7.43 (m, 4H), 4.8 (m, 1H), 4.2 (t, J=9 Hz, 1H), 3.86 (dd, J=6.6, 9.3 Hz, 1H), 3.57 (d, J=5.1 Hz, 2H), 3.56 (s, 2H), 2.29 (s, 6H), 1.97 (s, 3H).

It will be evident to one skilled in the art that the invention is not limited to the forgoing examples, and that it can be embodied in other specific forms without departing from the essential attributes thereof. Thus, it is desired that the examples be considered as illustrative and not restrictive, reference being made to the claims, and that all changes which come within the meaning and range of equivalency of the claims be embraced therein.

What is claimed is:

1. A compound of formula (I) 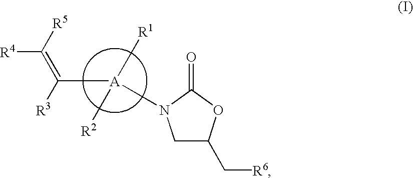
or a therapeutically acceptable salt or prodrug thereof, wherein
A is selected from
(a) phenyl,
(b) a five-membered aromatic ring containing one or two atoms selected from N, O, and S, and the remaining atoms are carbon,
wherein the groups defining (b) are substituted on a substitutable carbon or nitrogen atom in the ring, and
(c) a six-membered aromatic ring containing one or two nitrogen atoms, and the remaining atoms are carbon;
wherein the groups defining (c) are substituted on a substitutable carbon atom in the ring;
R¹ and R² are independently selected from hydrogen, alkyl, alkoxy, thioalkoxy, cycloalkyl, hydroxy, amino, aminoalkyl, halo, haloalkyl, and perfluoroalkyl;
R³, R⁴, and R⁵ are independently selected from
(a) hydrogen,
(b) carboxamido, cyano, halo, nitro, perfluoroalkyl,
(c) alkyl, alkanoyl, cycloalkyl, cyclothioalkoxy, cycloalkylsulfinyl, cycloalkoxycarbonyl, thioalkoxy, alkylsulfinyl, alkylsulfonyl, alkoxycarbonyl, cycloalkenyl, thiocycloalkenyloxy, cycloalkenylsulfinyl, cycloalkenylsulfonyl,
wherein the groups defining (c) can be optionally substituted with 1-5 substituents independently selected from alkoxy, alkanoyloxy, alkoxycarbonyl, amino, azido, carboxamido, carboxy, cyano, halo, hydroxy, nitro, perfluoroalkyl, perfluoroalkoxy, oxo, thioalkoxy, unsubstituted or substituted aryl, unsubstituted or substituted heteroaryl, and unsubstituted or substituted heterocycle,
(d) aryl, arylalkyl, arylthio, arylsulfinyl, arylsulfonyl, aryloxycarbonyl, heteroaryl, heteroarylalkyl, heteroarylsulfinyl, heteroarylsulfonyl, heteroaryloxycarbonyl, heterocycle, (heterocycle)alkyl, (heterocycle)sulfinyl, (heterocycle)sulfonyl, and (heterocycle)oxycarbonyl,
wherein the groups defining (d) can be optionally substituted with 1-5 substituents independently selected from alkyl, alkoxy, alkoxyalkyl, alkoxyalkenyl, alkanoyl, alkanoyloxy, alkanoyloxyalkyl, alkanoyloxyalkenyl, alkoxycarbonyl, alkoxycarbonylalkyl, alkoxycarbonylalkenyl, alkylsulfonyl, alkylsulfonylalkyl, alkylsulfonylalkenyl, amino, aminoalkyl, aminoalkenyl, aminosulfonyl, aminosulfonylalkyl, aminosulfonylalkenyl, azido, carboxaldehyde, (carboxaldehyde)alkyl, (carboxaldehyde)alkenyl, carboxamido, carboxamidoalkyl, carboxamidoalkenyl, carboxy, carboxyalkyl, carboxyalkenyl, cyano, cyanoalkyl, cyanoalkenyl, halo, haloalkyl, baloalkenyl, hydroxy, hydroxyalkyl, hydroxyalkenyl, nitro, oxo, perfluoroalkyl, perfluoroalkoxy, perfluoroalkoxyalkyl, perfluoroalkoxyalkenyl thioalkoxy, thioalkoxyalkyl, thioalkoxyalkenyl, unsubstituted or substituted aryl, unsubstituted or substituted heteroaryl, and unsubstituted or substituted heterocycle,
wherein for the groups defining (c) and (d), the substituted aryl, the substituted heteroaryl, and the substituted heterocycle are substituted with 1-5 substituents independently selected from alkyl, alkoxy, carboxy, azido, carboxaldehyde, halo, hydroxy, cyano, thioalkoxy, amino, alkoxycarbonyl, arylsulfonyl, oxo, perfluoroalkyl, and perfluoroalkoxy; or
wherein R⁴ and R5 together are a 3- to 8-membered cycloalkyl;
with the proviso that combinations wherein R³, R⁴, and R⁵ are hydrogen are excluded;
R⁶ is selected from NHR⁷, N-phthalimide, NR⁷R⁸, N(R⁸)C(O)OR⁹, N(R⁸)C(O)N(R⁸)₂, OR⁹, SR⁹, S(O)R⁹, and SO₂R⁹;
R⁷ is selected from alkanoyl, aryloyl, thioalkanoyl, heteroaryl, heteroarylalkyl, (heteroaryl)oyl, heterocycle, and (heterocycle)alkyl,
wherein the groups defining R⁷ can be optionally substituted with 1-5 substituents independently selected from alkyl, alkoxy, alkoxycarbonyl, carboxy, azido, carboxaldehyde, halo, hydroxy, perfluoroalkyl, and perfluoroalkoxy;
R⁸ is selected from
(a) hydrogen,
(b) alkyl,
wherein the alkyl can be optionally substituted with 1-5 substituents independently selected from alkoxy, aryl, alkoxycarbonyl, carboxy, azido, carboxaldehyde, halo, hydroxy, perfluoroalkyl, and perfluoroalkoxy;
(c) cycloalkyl, cycloalkylalkyl, aryl, heteroaryl, heteroarylalkyl, heterocycle, and (heterocycle)alkyl;
wherein the groups defining (c) can be optionally substituted with 1-5 substituents independently selected from alkyl, alkoxy, carboxy, azido, carboxaldehyde, halo, hydroxy, perfluoroalkyl, and perfluoroalkoxy; and
(d) a nitrogen protecting group; and
R⁹is selected from
(a) alkyl,
wherein the alkyl can be optionally substituted with 1-5 substituents independently selected from alkoxy, carboxy, azido, carboxaldehyde, halo, hydroxy, perfluoroalkyl, and perfluoroalkoxy,
(b) cycloalkyl, cycloalkylalkyl, aryl, heteroaryl, heteroarylalkyl, heterocycle, and (heterocycle)alkyl;
wherein the groups defining (b) can be optionally substituted with 1-5 substituents independently selected from alkyl, alkoxy, carboxy, azido, carboxaldehyde, halo, hydroxy, perfluoroalkyl, and perfluoroalkoxy;
with the proviso that combinations wherein A is phenyl, R⁶ is NHR⁷ wherein R⁷ is alkanoyl, one of R³, R⁴ or R⁵ is hydrogen, another is selected from hydrogen, perfluoroalkyl, or unsubstituted alkyl, and the remainder is phenyl unsubstituted or substituted with at least one halo substituent, are excluded; and
with the proviso that combinations wherein A is phenyl, R⁶ is NHR⁷ wherein R⁷ is alkanoyl, one of R³, R⁴ or R⁵ is cyano, carboxaldehyde, or nitro, and the remainder are hydrogen or alkyl are excluded.

2. A compound set forth in claim 1 of formula (II) 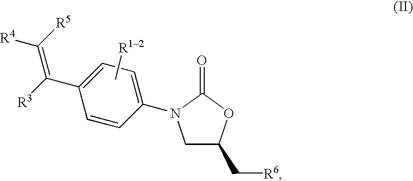
or a therapeutically acceptable salt or prodrug thereof, wherein R¹, R², R³, R⁴, R⁵ and R⁶ are defined therein.

3. A compound set forth in claim 1 wherein R¹ and R² are hydrogen.

4. A compound set forth in claim 1 wherein R¹ is hydrogen and R² is halo.

5. A compound set forth in claim 1 wherein R³ is hydrogen.

6. A compound set forth in claim 1 wherein R³ is halo.

7. A compound set forth in claim 1 wherein R⁴ is hydrogen.

8. A compound set forth in claim 1 wherein R⁴ is alkyl.

9. A compound set forth in claim 1 wherein R⁴ is halo.

10. A compound set forth claim 1 wherein R⁴ is aryl wherein the aryl is substituted as set forth therein.

11. A compound set forth in claim 1 wherein R⁴ is heteroaryl wherein the heteroaryl is substituted as set forth therein.

12. A compound set forth in claim 1 wherein R⁴ is arylsulfonyl.

13. A compound set forth in claim 1 wherein R⁵ is alkoxycarbonyl.

14. A compound set forth in claim 1 wherein R⁶ is —NHR⁷ and R⁷ is defined therein.

15. A compound set forth in claim 14 wherein R⁷ is alkanoyl.

16. A composition comprising a compound of formula (I), or a therapeutically acceptable salt or prodrug thereof, and a therapeutically acceptable excipient.

17. A composition comprising a compound of formula (II), or a therapeutically acceptable salt or prodrug thereof, and a therapeutically acceptable excipient.

18. A method for treating bacterial infections in a patient comprising administering to the patient a therapeutically acceptable amount of a compound of formula (I), or a therapeutically acceptable salt or prodrug thereof.

19. A method for treating bacterial infections in a patient comprising administering to the patient a therapeutically acceptable amount of a compound of formula (II), or a therapeutically acceptable salt or prodrug thereof.

20. A method for treating psoriasis in a patient comprising administering to the patient a therapeutically acceptable amount of a compound of formula (I), or a therapeutically acceptable salt or prodrug thereof.

21. A method for treating arthritis in a patient comprising administering to the patient a therapeutically acceptable amount of a compound of formula (I), or a therapeutically acceptable salt or prodrug thereof.

22. A method for treating toxicity due to chemotherapy in a patient comprising administering to the patient a therapeutically acceptable amount of a compound of formula (I), or a therapeutically acceptable salt or prodrug thereof.

23. A compound selected from
N-(((5S)-3-(4-((E)-2-(4-cyanophenyl)ethenyl)phenyl)-2-oxo-1,3-oxazolidin-5-yl)methyl)acetamide,
ethyl (2Z)-3-(4-((5S)-5-((acetylamino)methyl)-2-oxo-1,3-oxazolidin-3-yl)phenyl)-2-fluoro-2-propenoate,
N-(((5S)-3-(4-((Z)-2-bromo-2-(3-pyridinyl)ethenyl)-3-fluorophenyl)-2-oxo-1,3-oxazolidin-5-yl)methyl)acetamide,
N-(((5S)-3-(4-((Z)-2-bromo-2-(2-furyl)ethenyl)-3-fluorophenyl)-2-oxo-1,3-oxazolidin-5-yl)methyl)acetamide,
N-(((5S)-3-(4-((Z)-2-bromo-2-(3,5-dimethyl-4-isoxazolyl)ethenyl)-3-fluorophenyl)-2-oxo-1,3-oxazolidin-5-yl)methyl)acetamide,
N-(((5S)-3-(4-((Z)-2-(5-acetyl-2-thienyl)-2-bromo-ethenyl)-3-fluorophenyl)-2-oxo-1,3-oxazolidin-5-yl)methyl)-acetamide,
N-(((5S)-3-(4-((Z)-2-bromo-2-(3-cyanophenyl)ethenyl)-3-fluorophenyl)-2-oxo-1,3-oxazolidin-5-yl)methyl)acetamide,
N-(((5 S)-3-(4-((Z)-2-bromo-2-(3-formylphenyl)ethenyl)-3-fluorophenyl)-2-oxo-1,3-oxazolidin-5-yl)methyl)acetamide,
N-(((5S)-3-(4-((Z)-2-bromo-2-(3-(hydroxymethyl)phenyl)ethenyl)-3-fluorophenyl)-2-oxo-1 ,3-oxazolidin-5-yl)methyl)acetamide,
N-(((5S)-3-(4-((Z)-2-bromo-2-(4-(hydroxymethyl)phenyl)ethenyl)-3-fluorophenyl)-2-oxo-1,3-oxazolidin-5-yl)methyl)acetamide,
N-(((5S)-3-(4-((Z)-2-(3-acetylphenyl)-2-bromoethenyl)-3-fluorophenyl)-2-oxo-1,3-oxazolidin-5-yl)methyl)acetamide,
N-(((5S)-3-(4-((Z)-2-bromo-2-(4-cyanophenyl)ethenyl)-3-fluorophenyl)-2-oxo-1,3-oxazolidin-5-yl)methyl)acetamide,
N-(((5S)-3-(4-((Z)-2-bromo-2-(4-isoquinolinyl)ethenyl)-3-fluorophenyl)-2-oxo-1,3-oxazolidin-5-yl)methyl)acetamide,
N-(((5S)-3-(4-((Z)-2-bromo-2-(4-pyridinyl)ethenyl)-3-fluorophenyl)-2-oxo-1,3-oxazolidin-5-yl)methyl)acetamide,
methyl 3-((E)-2-(4-((5S)-5-((acetylamino)methyl)-2-oxo-1 ,3-oxazolidin-3-yl)-2-fluorophenyl)ethenyl)benzoate,
(2E)-3-(4-((Z)-2-(4-((5S)-5-((acetylamino)methyl)-2-oxo-1,3-oxazolidin-3-yl)-2-fluorophenyl)-1-bromoethenyl)phenyl)-2-propenoic acid,
methyl 3-((Z)-2-(4-((5S)-5-((acetylamino)methyl)-2-oxo-1,3-oxazolidin-3-yl)-2-fluorophenyl)-1-bromoethenyl)benzoate,
N-(((5S)-3-(4-((Z)-2-(4-acetylphenyl)-2-bromoethenyl)-3-fluorophenyl)-2-oxo-1,3-oxazolidin-5-yl)methyl)acetamide,
N-(((5S)-3-(3-fluoro-4-((E)-2-fluoro-2-(phenylsulfonyl)ethenyl)phenyl)-2-oxo-1,3-oxazolidin-5-yl)methyl)acetamide,
N-(((5S)-3-(4-((Z)-2-bromo-2-(4-methoxy-3-pyridinyl)ethenyl)-3-fluorophenyl)-2-oxo-1,3-oxazolidin-5-yl)methyl)acetamide,
N-(3-((Z)-2-(4-((5S)-5-((acetylamino)methyl)-2-oxo-1,3-oxazolidin-3-yl)-2-fluorophenyl)-1-bromoethenyl)phenyl)acetamide;
N-(((5S)-3-(3-fluoro-4-((E)-2-pyridin-3-ylethenyl)phenyl)-2-oxo-1,3-oxazolidin-5-yl)methyl)acetamide;
N-(((5S)-3-(4-((E)-2-(5-cyanopyridin-3-yl)ethenyl)-3-fluorophenyl)-2-oxo-1,3-oxazolidin-5-yl)methyl)acetamide;
N-(((5S)-3-(4-((1E)-2-(5-cyanopyridin-3-yl)prop-1-enyl)-3-fluorophenyl)-2-oxo-1,3-oxazolidin-5-yl)methyl)acetamide;
N-(((5S)-3-(4-((1Z)-1-(5-cyanopyridin-3-yl)prop-1-enyl)-3-fluorophenyl)-2-oxo-1,3-oxazolidin-5-yl)methyl)acetamide;
(5S)-3-(4-((Z)-2-bromo-2-pyridin-3-ylethenyl)-3-fluorophenyl)-5-((isoxazol-3-ylamino)methyl)-1,3-oxazolidin-2-one;
N-(((5S)-3-(3-fluoro-4-((Z)-2-fluoro-2-pyridin-3-ylethenyl)phenyl)-2-oxo-1,3-oxazolidin-5-yl)methyl)acetamide;
N-(((5 S)-3-(3-fluoro-4-((E)-2-fluoro-2-pyridin-3-ylethenyl)phenyl)-2-oxo-1,3-oxazolidin-5-yl)methyl)acetamide;
N-(((5S)-3-(3-fluoro-4-((Z)-2-fluoro-2-pyrimidin-5-ylethenyl)phenyl)-2-oxo-1,3-oxazolidin-5-yl)methyl)acetamide;
N-(((5S)-3-(3-fluoro-4-((Z)-2-fluoro-2-( 1,3-thiazol-2-yl)ethenyl)phenyl)-2-oxo-1,3-oxazolidin-5-yl)methyl)acetamide;
N-(((5S)-3-(4-((Z)-2-(2-aminopyrimidin-5-yl)-2-fluoroethenyl)-3-fluorophenyl)-2-oxo-1,3-oxazolidin-5-yl)methyl)acetamide;
N-(((5S)-3-(3-fluoro-4-((Z)-2-fluoro-2-(2-methyl-2H-tetraazol-5-yl)ethenyl)phenyl)-2-oxo-1,3-oxazolidin-5-yl)methyl)acetamide;
N-(((5S)-3-(4-((Z)-2-(2-cyanothien-3-yl)-2-fluoroethenyl)-3-fluorophenyl)-2-oxo-1,3-oxazolidin-5-yl)methyl)acetamide;
N-(((5S)-3-(4-((Z)-2-(5-cyanopyridin-3-yl)-2-fluoroethenyl)-3-fluorophenyl)-2-oxo-1,3-oxazolidin-5-yl)methyl)acetamide;
N-(((5S)-3-(4-((Z)-2-(3-acetylphenyl)-2-fluoroethenyl)-3-fluorophenyl)-2-oxo-1,3-oxazolidin-5-yl)methyl)acetamide;
N-(((5S)-3-(3-fluoro-4-((Z)-2-fluoro-2-(3-formylphenyl)ethenyl)phenyl)-2-oxo-1,3-oxazolidin-5-yl)methyl)acetamide;
N-(((5S)-3-(4-((Z)-2-(3-cyanophenyl)-2-fluoroethenyl)-3-fluorophenyl)-2-oxo-1,3-oxazolidin-5-yl)methyl)acetamide;
N-(((5S)-3-(3-fluoro-4-((E/Z)-2-fluoroethenyl)phenyl)-2-oxo-1,3-oxazolidin-5-yl)methyl)acetamide;
N-(((5S)-3-(4-((Z)-2-(3-aminophenyl)-2-bromoethenyl)-3-fluorophenyl)-2-oxo-1,3-oxazolidin-5-yl)methyl)acetamide;
N-(((5S)-3-(4-((Z)-2-bromo-2-(5-cyanopyridin-3-yl)ethenyl)-3-fluorophenyl)-2-oxo-1,3-oxazolidin-5-yl)methyl)acetamide;
methyl 3-((Z)-2-(4-((5S)-5-((acetylamino)methyl)-2-oxo-1,3-oxazolidin-3-yl)-2-fluorophenyl)-1-bromoethenyl)-5-aminobenzoate;
N-(((5S)-3-(4-((Z)-2-(3-(aminomethyl)phenyl)-2-bromoethenyl)-3-fluorophenyl)-2-oxo-1,3-oxazolidin-5-yl)methyl)acetamide;
N-(((5S)-3-(4-((Z)-2-bromo-2-phenylethenyl)-3-fluorophenyl)-2-oxo-1,3-oxazolidin-5-yl)methyl)acetamide;
3-((Z)-2-(4-((5S)-5-((acetylamino)methyl)-2-oxo-1,3-oxazolidin-3-yl)-2-fluorophenyl)-1-bromoethenyl)benzoic acid;
3-((Z)-2-(4-((5S)-5-((acetylamino)methyl)-2-oxo-1,3-oxazolidin-3-yl)-2-fluorophenyl)-1-bromoethenyl)benzamide; and
N-(((5S)-3-(4-((Z)-2-bromo-2-(3-((dimethylamino)methyl)phenyl)ethenyl)-3-fluorophenyl)-2-oxo-1,3-oxazolidin-5-yl)methyl)acetamide.
